(19) United States
(12) Patent Application Publication
(10) Pub. No.: US 2024/0043937 A1
(43) Pub. Date: Feb. 8, 2024

(54) METHOD FOR DETERMINING SUBTYPE OF PANCREATIC DUCTAL ADENOCARCINOMA, AND SUBTYPE DETERMINATION KIT

(71) Applicant: BERTIS INC, Gyeonggi-do (KR)

(72) Inventors: Sangwon LEE, Seoul (KR); Jingi BAE, Daejeon (KR); Dowoon NAM, Seoul (KR); Daehee HWANG, Suwon-si (KR); Jin-Young JANG, Seoul (KR); Cheolju LEE, Seoul (KR); Sungho SHIN, Seoul (KR); Min-Sik KIM, Daegu (KR)

(73) Assignee: BERTIS INC, Gyeonggi-do (KR)

(21) Appl. No.: 18/268,858

(22) PCT Filed: Aug. 26, 2021
(86) PCT No.: PCT/KR2021/011417

§ 371 Date: Jun. 21, 2023

(30) Foreign Application Priority Data
Dec. 22, 2020 (KR) 10-2020-0181254
Aug. 20, 2021 (KR) 10-2021-0110508

(51) Int. Cl. C12Q 1/6886; C12Q 1/6806

(52) U.S. Cl. CPC C12Q 1/6886; C12Q 1/6806; G01N 2800/56; G01N 2800/7028

(57) ABSTRACT

The present invention relates to a method of determining the subtype of a pancreatic ductal adenocarcinoma patient through proteogenomic analysis of PDAC. The method of determining the subtype of pancreatic cancer according to one embodiment of the present invention comprises steps of: (1) pulverizing a pancreatic ductal adenocarcinoma lesion tissue isolated from a pancreatic ductal adenocarcinoma patient; (2) obtaining a peptide sample for the patient by extracting and digesting proteins from the lesion tissue; (3) measuring the expression levels of representative genes of pancreatic ductal adenocarcinoma subtypes 1 to 6 from the peptide sample for the patient; and (4) determining the subtype of the pancreatic ductal adenocarcinoma patient by comparing the expression levels of the representative genes of pancreatic ductal adenocarcinoma subtypes 1 to 6.

### TECHNICAL FIELD

The present invention relates to a method and a kit for determining the subtype of pancreatic ductal adenocarcinoma, and more particularly, to a method and a kit for determining the subtype of pancreatic ductal adenocarcinoma in a patient using information on the subtype of stratified pancreatic ductal adenocarcinoma by proteogenomic analysis through integration of genomic, mRNA and proteomic data.

### BACKGROUND ART

In Korea, the incidence of pancreatic cancer ranks 9^{th} among all cancers, but the mortality rate thereof ranks 5^{th} as most of patients diagnosed with pancreatic cancer die. In the United States, pancreatic cancer is currently the fourth leading cause of cancer-related death and is predicted to become the second leading cause of cancer-related death in the United States by 2030. Pancreatic cancer can be cured only by surgery because there is no very effective systemic treatment method, but due to its anatomical characteristics, it invades major blood vessels or metastasizes systemically, and thus is found in 80% of patients in a state in which a cure is impossible. Even if surgery and chemotherapy are actively performed in patients within stage 2 where surgery is possible (around 20% of all patients), recurrence occurs in about 70% of patients, and the 5-year survival rate is only about 20%, indicating that pancreatic cancer is the most incurable tumor. In other words, only about 5 to 8% of all patients with pancreatic cancer can be cured, and more than 90% of the remaining patients have tumors that are refractory to both current treatment methods such as surgery and chemotherapy. Accordingly, efforts are desperately needed to overcome pancreatic cancer through studies on the mechanism of pancreatic cancer and selective treatment using the same.

Traditionally, chemotherapy based on 5-fluorouracil (5-FU) or gemcitabine is performed for pancreatic cancer, but the response rate is low, and there is no anticancer drug that consistently shows a clear effect. In addition, clinical diagnostic methods, such as imaging and pathological examinations, cannot predict treatment responsiveness/resistance, the possibility of early recurrence, and prognosis. Therefore, there is a need for a novel approach that can classify pancreatic cancer according to its biological mechanism and predict appropriate treatment and prognosis based on the classification.

According to the results of recent proteogenomic studies on various cancer diseases, integrated proteogenomic data provide more precise information on cancer subtypes than genomic data, and provide more complete information on the pathogenesis of cancer for each subtype. Therefore, even for pancreatic cancer, it is possible to determine the subtype of a pancreatic cancer patient by the pancreatic cancer subtype determination technology based on the pathogenesis of each subtype based on proteogenomic data, thereby developing a precision medical technology for pancreatic cancer that can provide optimal treatment for each subtype through the development of subtype-specific therapeutic agents in the future. For example, Patent Document 1 discloses a method for determining subtypes of pancreatic tumors, wherein the subtypes of pancreatic cancer are classified as four subtypes using TPI1, GAPDH, ENO1, LDHA, and PGK1.

### PRIOR ART DOCUMENTS

### Patent Documents

(Patent Document 0001) International Patent Publication No. WO2020-205993 (Oct. 8, 2020)

### DISCLOSURE

### Technical Problem

The present invention is intended to provide a method and a kit for determining the subtype of pancreatic ductal adenocarcinoma in a patient using information on the subtype of stratified pancreatic ductal adenocarcinoma.

### Technical Solution

One embodiment of the present invention provides a method for determining the subtype of pancreatic ductal adenocarcinoma, the method comprising the following steps (1) to (4):
(1) pulverizing a pancreatic ductal adenocarcinoma lesion tissue isolated from a pancreatic ductal adenocarcinoma patient;
(2) obtaining a peptide sample for the patient by extracting and digesting proteins from the lesion tissue;
(3) measuring the expression levels of representative genes of pancreatic ductal adenocarcinoma subtypes 1 to 6 from the peptide sample for the patient, wherein the representative genes of pancreatic ductal adenocarcinoma subtypes 1 to 6 are each at least one selected from the group consisting of the following genes:
representative genes of subtype 1 (Sub1): CLDN18, EPS8L3, CAPN5, GMDS, BCAS1, IDH1, DDAH1, SOD1, VIL1, GPX2, AOC1, LGALS4, MICU2, POF1B, MICU1, PLS1, and BDH1;
representative genes of subtype 2 (Sub2): UNC5B, PPP1R3G, IGFBP3, EDIL3, CLSTN1, COL11A1, P4HA1, PDLIM4, ST5, FSTL1, PPP1R13L, PLTP, PDLIM7, and CALU;
representative genes of subtype 3 (Sub3): MYH9, FLNA, P4HA2, LOXL2, FN1, CD55, FLT1, ECM1, CCDC80, TSKU, HTRA1, COL12A1, SPON2, and ANGPTL2;
representative genes of subtype 4 (Sub4): PLEC, LPGAT1, NRDC, PRPF40A, CSDE1, IPO7, CDK1, HMGA1, DDX5, RASA1, ADSS, GMPS, CSE1L, PSME3, CAPRIN1, and BZW1;
representative genes of subtype 5 (Sub5): HSPB6, HSPA12A, ANXA6, VIM, UCHL1, PRPH, MAP1B, CD81, ANK2, AKAP12, ITSN1, RTN1, COL28A1, KCTD12, SPON1, SYNPO2, and EPB41L3; and
representative genes of subtype 6 (Sub6): CTNND2, DTNA, REG1A, PRSS2, CPA1, CPB1, ACAT1, CPA2, PNLIPRP1, PRDX4, SNTB1, PDCD4, CTRC, FKBP11, and SEC11C; and
(4) determining the subtype of the pancreatic ductal adenocarcinoma patient by comparing the expression levels of the representative genes of pancreatic ductal adenocarcinoma subtypes 1 to 6.

Another embodiment of the present invention provides a kit for determining the subtype of pancreatic ductal adenocarcinoma. The kit for determining the subtype of pancreatic ductal adenocarcinoma may comprise agents for measuring the expression levels of representative genes of pancreatic ductal adenocarcinoma subtypes 1 to 6, wherein the representative genes of pancreatic ductal adenocarcinoma subtypes 1 to 6 may each be at least one selected from the group consisting of the following genes:
representative genes of subtype 1 (Sub1): CLDN18, EPS8L3, CAPN5, GMDS, BCAS1, IDH1, DDAH1, SOD1, VIL1, GPX2, AOC1, LGALS4, MICU2, POF1B, MICU1, PLS1, and BDH1;
representative genes of subtype 2 (Sub2): UNC5B, PPP1R3G, IGFBP3, EDIL3, CLSTN1, COL11A1, P4HA1, PDLIM4, ST5, FSTL1, PPP1R13L, PLTP, PDLIM7, and CALU;
representative genes of subtype 3 (Sub3): MYH9, FLNA, P4HA2, LOXL2, FN1, CD55, FLT1, ECM1, CCDC80, TSKU, HTRA1, COL12A1, SPON2, and ANGPTL2;
representative genes of subtype 4 (Sub4): PLEC, LPGAT1, NRDC, PRPF40A, CSDE1, IPO7, CDK1, HMGA1, DDX5, RASA1, ADSS, GMPS, CSE1L, PSME3, CAPRIN1, and BZW1;
representative genes of subtype 5 (Sub5): HSPB6, HSPA12A, ANXA6, VIM, UCHL1, PRPH, MAP1B, CD81, ANK2, AKAP12, ITSN1, RTN1, COL28A1, KCTD12, SPON1, SYNPO2, and EPB41L3; and
representative genes of subtype 6 (Sub6): CTNND2, DTNA, REG1A, PRSS2, CPA1, CPB1, ACAT1, CPA2, PNLIPRP1, PRDX4, SNTB1, PDCD4, CTRC, FKBP11, and SEC11C.

Another embodiment of the present invention provides a method for predicting the prognosis of a pancreatic ductal adenocarcinoma patient, the method comprising the following steps (1) to (5):
(1) pulverizing a pancreatic ductal adenocarcinoma lesion tissue isolated from a pancreatic ductal adenocarcinoma patient;
(2) obtaining a peptide sample for the patient by extracting and digesting proteins from the lesion tissue;
(3) measuring the expression levels of representative genes of pancreatic ductal adenocarcinoma subtypes 1 to 6 from the peptide sample for the patient, wherein the representative genes of pancreatic ductal adenocarcinoma subtypes 1 to 6 are each at least one selected from the group consisting of the following genes:
representative genes of subtype 1 (Sub1): CLDN18, EPS8L3, CAPN5, GMDS, BCAS1, IDH1, DDAH1, SOD1, VIL1, GPX2, AOC1, LGALS4, MICU2, POF1B, MICU1, PLS1, and BDH1;
representative genes of subtype 2 (Sub2): UNC5B, PPP1R3G, IGFBP3, EDIL3, CLSTN1, COL11A1, P4HA1, PDLIM4, ST5, FSTL1, PPP1R13L, PLTP, PDLIM7, and CALU;
representative genes of subtype 3 (Sub3): MYH9, FLNA, P4HA2, LOXL2, FN1, CD55, FLT1, ECM1, CCDC80, TSKU, HTRA1, COL12A1, SPON2, and ANGPTL2;
representative genes of subtype 4 (Sub4): PLEC, LPGAT1, NRDC, PRPF40A, CSDE1, IPO7, CDK1, HMGA1, DDX5, RASA1, ADSS, GMPS, CSE1L, PSME3, CAPRIN1, and BZW1;
representative genes of subtype 5 (Sub5): HSPB6, HSPA12A, ANXA6, VIM, UCHL1, PRPH, MAP1B, CD81, ANK2, AKAP12, ITSN1, RTN1, COL28A1, KCTD12, SPON1, SYNPO2, and EPB41L3; and
representative genes of subtype 6 (Sub6): CTNND2, DTNA, REG1A, PRSS2, CPA1, CPB1, ACAT1, CPA2, PNLIPRP1, PRDX4, SNTB1, PDCD4, CTRC, FKBP11, and SEC11C;
(4) determining the subtype of the pancreatic ductal adenocarcinoma patient by comparing the expression levels of the representative genes of pancreatic ductal adenocarcinoma subtypes 1 to 6; and
(5) predicting prognosis of the patient based on the determined subtype.

### Advantageous Effects

The proteogenomic analysis according to one embodiment of the present invention can improve understanding of PDAC and stratification of PDAC patients, and improve treatment of pancreatic cancer patients by determining pancreatic ductal adenocarcinoma subtypes.

According to one embodiment of the present invention, it is possible to determine subtypes of pancreatic ductal adenocarcinoma patients through proteogenomic analysis of PDAC. This will enable precision medical technology for pancreatic cancer that can provide optimal treatment for each subtype through the development of subtype-specific therapeutic agents in the future.

According to one embodiment of the present invention, it is possible to predict prognosis by determining the subtype of pancreatic cancer, and to develop subtype-specific new drugs.

### BRIEF DESCRIPTION OF DRAWINGS

FIGS. 1(A) to and 2(I) relate to the correlation between mutation-protein abundance and phosphorylation in apoptosis and actin cytoskeleton pathways in PDAC.

FIG. 1(A) shows mutations per megabase in individual patients (top), SMG mutation types in each patient (middle right); mutation frequency for each gene in patients (middle left); and clinical parameters for each patient (bottom). FIG. 1(B) shows comparisons of somatic mutation frequencies of SMG. Red labeling indicates that the gene was detected at a significantly (p<0.05 by proportional test) higher frequency in the cohort of the present invention than in the other cohorts. FIG. 1(C) shows phosphopeptides whose expression has been upregulated in tumors having somatic mutations in KRAS, SMAD4 and ARID1A. The colored bar represents the gradient of loge-fold-changes for intensity levels for phosphopeptides relative to their mean levels.

FIG. 1(D) shows cellular pathways associated with proteins for which phosphorylation levels were correlated with somatic mutations in KRAS, SMAD4 and ARID1A. The heat map shows the enrichment significance of each pathway by protein which mutation-phosphorylation correlations were identified. Significance is displayed as −log₁₀ (p), where p is the p value for enrichment. FIGS. 1(E) to 1(G) show the correlation between somatic mutations and protein abundance or phosphorylation levels in TP53 (E), RB1 (F) and ATM (G). The lollipop plots show somatic mutations (circles) and phosphorylation sites (triangles) detected in the gene structure (top). The height of the lollipop indicates the number of patients with the corresponding mutations, and colors represent the mutation types (see legend in A). Samples are sorted based on somatic mutations. Protein abundance or intensity of phosphopeptides normalized relative to their median levels across all patients are shown in bar plots (bottom; red and blue, higher and lower than the median, respectively). Enriched mutation sites are indicated by arrows. FIG. 1(H) shows network model describing interactions between EMT-related proteins and strong correlations (orange node) of their mutation phosphorylation in KRAS, SMAD4 or ARID1A. Gray nodes indicate molecules added in the pathways to increase connections between the nodes but having no significant mutation-phosphorylation correlations. Solid arrows indicate direct activations; dotted arrows indicate indirect activations; gray lines indicate protein-protein interactions; thick lines indicate plasma membrane. FIG. 1(I) shows the correlation of somatic mutations with protein abundance and phosphorylation levels in ARID1A.

FIG. 2(A) shows a workflow for proteogenomic analysis of PDAC. Exome-sequencing analysis of cancer tissues and blood samples and RNA-sequencing of cancer tissues were performed for tumors from 196 patients while mass spectrometry-based proteomic analyses (global proteome and phosphoproteome) were performed for tumors from 150 patients. Distribution of tumor cellularity was shown for tumors from 196 patients (bottom left). The tumors with cellularity >15% were used for proteogenomic analysis, and tumors with cellularity >19% were used for proteomic analysis. FIG. 2(B) shows the numbers of non-redundant peptides identified from global proteome and phosphoproteome data. FIG. 2(C) shows the numbers of protein-coding genes identified from mRNA sequencing and proteome data (global proteome and phosphoproteome). The average numbers of peptides and protein-coding genes are indicated in (B) and (C), respectively. FIG. 2(D) shows the numbers of somatic mutations altering protein sequences and genes carrying the mutations identified from exome-sequencing data. FIG. 2(E) shows relationships among significantly mutated genes (SMGs) identified from the cohort of the present invention and previous cohorts of the TCGA (Cancer Genome Atlas Research Network. Electronic address and Cancer Genome Atlas Research, 2017), Bailey et al., 2016, Biankin et al., 2012, Witkiewicz et al., 2015), and Waddell et al., 2015. FIGS. 2(F) to 2(H) show the association of somatic mutations with protein abundance or phosphopeptide level in KMT2D (F), AHNAK2 (G) and FCGBP (H). For each gene, lollipop plots (top) show the detected somatic mutations (circles) and phosphorylation sites (triangles). The height of the lollipop indicates the number of patients with the corresponding mutations, and colors indicate the mutation types (see legend). Protein abundance or intensity of the phosphopeptides was normalized relative to their median levels across all patients displayed in bar plots (bottom; red and blue, higher and lower than the median, respectively). FIG. 2(I) shows correlations of copy number variations (CNVs) with mRNA (left) and protein (right) expression levels. Red and blue indicate positive and negative correlations, respectively. Diagonal and off-diagonal elements in the heat maps indicate cis- and trans-correlations between CNVs and expression levels of mRNA or protein. Along the chromosome, the numbers of CNVs that correlated specifically with either mRNA or protein expression levels and commonly with both mRNA and protein expression levels were displayed in blue (top) and black (bottom) bars, respectively.

FIGS. 3(A) to 4(G) relate to mRNA-protein abundance correlations and new oncogene and tumor suppressor candidates.

FIG. 3(A) shows distributions of survival differences (Chi-square statistic values) for the genes with significant (FDR <0.01) and non-significant (FDR>0.1) mRNA—protein correlations. p<0.01, Student's t-test. In violin plots, the line indicates the median value. FIG. 3(B) shows cellular pathways represented by the genes showing positive and negative mRNA-survival correlations. The enrichment significance of each pathway is indicated as—log₁₀ (p), where p is the p-value for enrichment. Red dotted lines: p=0.05. FIG. 3(C) shows selection of oncogene and tumor suppressor candidates. Among genes with significant (FDR <0.01) mRNA-protein correlations, 12 tumor suppressor and 19 oncogene candidates were identified with significant positive (hazard ratio<1) and negative (hazard ratio>1) mRNA-survival correlations in all four PDAC cohorts, respectively. According to the criteria (FIG. 7(D)), 6 tumor suppressors and 4 oncogenes were selected for further study. FIGS. 3D and 3E show AsPC1 counts (D) upon expression of control shRNA (shCtr1) or indicated shRNAs targeting oncogene candidates to induce knockdown (n≥3/condition), and cell count determined on day 5 (E). FIG. 3(F) and 3(G) show representative images (F) and quantification (G) of wound healing measured at 0 or 48 hours after wounding by scratching AsPC1 cells transfected with the indicated siRNAs (n≥2/condition). Wound healing was quantified at 48 hours. FIGS. 3(H) and 3(I) show AsPC1 counts (H) after overexpression of the indicated tumor suppressor candidates (n≥2/condition). Cell numbers are determined on day 3 (I). Data are presented as mean±SEM. *, p<0.05; **, p<0.01; ***, p<0.001; ****, p<0.0001 by two-way (D and H) and one-way (E, G and I) analysis of variance (ANOVA) with Dunnett's post hoc correction.

FIG. 4(A) shows the distribution of Spearman's correlation coefficients of mRNA and protein abundances of individual genes across patients. Yellow and blue indicate positive and negative correlations, respectively. FIG. 4(B) shows differential association of the genes having high and low mRNA-protein abundance correlations with KEGG pathways. The genes involved in each KEGG pathway are indicated by yellow (positive correlation) and blue (negative correlation) bars. FIG. 4(C) shows cumulative density distributions of mRNA-protein correlations for the genes with significant (blue) and non-significant (red) survival differences between top 25% and bottom 25% of patients with the highest and the lowest mRNA expression levels, respectively. P<0.01 by Kolmogorov-Smirnov test. FIG. 4(D) shows selection of tumor suppressor and oncogene candidates for functional experiments. Among 12 tumor suppressor (TS) candidates and 19 oncogene candidates with significant positive (hazard ratio<1) and negative (hazard ratio>1) mRNA-survival correlations, respectively, in the four PDAC cohorts, the candidates with desirable survival curve patterns (red and blue lines, top and bottom 25% of patients with the highest and the lowest mRNA expression levels) in at least 3 PDAC cohorts were selected, resulting in 10 TS and 16 oncogene candidates. Among them, 9 TS and 7 oncogene candidates that have not previously been reported in pancreatic cancer were selected. Finally, among them, 7 TS and 5 oncogene candidates having expression levels larger than the median expression (log₂-FPKM=3.11) of expressed genes (FPKM>1) in AsPC1 cells were selected. Among them, IQGAP2 was excluded from functional experiments due to its large gene size, and KRT19 was excluded due to its involvement in the broad spectrum of functions. The mRNA expression profile of AsPC1 cells was obtained from Cancer Cell Line Encyclopedia (CCLE) (Ghandi et al., 2019). FPKM, fragments per kilobase of transcript per million. FIG. 4(E) shows relative mRNA expression levels of oncogene candidates in AsPC1 cells after knockdown, measured using quantitative RT-PCR analysis. mRNA levels were normalized by GAPDH, 18s RNA and HPRT (n=3 per condition), and then normalized to the resulting values of the target gene (e.g., shDCBLD2) by control shRNA-transduced cells (shCtr1). **, p<0.01; ***, p<0.001 by Student's t-test. FIGS. 4(F) and 4(G) show representative images obtained from immunoblotting (F) and relative protein levels of tumor suppressor candidates in AsPC1 cells after overexpression (G). The protein level of the target gene was normalized to that of a-actin.

FIGS. 5(A) to 6(D) relate to PDAC subtypes defined by proteogenomic analysis.

FIG. 5(A) shows RNA1-3 clusters defined by mRNA signatures (rna1-3) in the TCGA, PACA AU and PACA-CA cohorts. rna1-3 defining RNA1-3, respectively, are shown in the first heat map with numbers of mRNAs in rani-3 in parentheses. For each cohort, heat maps show the tumors classified into RNA1-3 based on rna1-3 (left) and the tumors that failed to correlate with rna1-3 (right). The subtype bar plot shows the subtypes that are predicted by molecular signatures as defined by Moffitt et al. 2015, Collisson et al., 2011), Bailey et al., 2016). FIG. 5(B) shows postdiagnosis survival of patients whose tumors belonged to RNA1-3 clusters in TCGA, PACA-AU and PACA-CA cohorts. FIGS. 5(C) and 5(D) show protein signatures (prot1-5 and phos1-4) that define Prot1-5 (C) and Phos1-4 (D) based on global proteome and phosphoproteome data. The numbers of proteins and phosphopeptides are indicated in parentheses. FIG. shows six subtypes (Sub1-6) identified by integrated clustering of mRNA, protein and phosphorylation data. The heat map shows indicator vectors for the corresponding clusters (rows) identified from clustering of individual types of data. Red, membership of individual samples belonging to the corresponding clusters; colored bar at top, Sub1-6; RNA1-3 cluster defined by rna1-3. FIG. 5(F) shows the survival durations of patients with tumors in Sub1-6. FIG. 5(G) shows the distribution of somatic mutations of SMGs in Sub1-6 and clinical parameters for each patient (bottom). FIG. 5(H) shows the distribution of mutations per megabase of somatic mutations identified from tumors in Sub1-6. In violin plots, the line indicates the median value. *, p<0.05 by one-way ANOVA with Sidak's post hoc correction.

FIG. 6(A) shows clustering results of mRNA sequencing data. In individual clustering results, cophenetic correlation coefficient plots (left) show how the coefficients vary with different numbers of clusters (k=2 to 6) and also when different numbers of the molecules selected with multiple percentages (10 to 30%) of median absolute deviations (MADs) were used for clustering. The silhouette width score plot (middle) shows core samples with positive scores in clusters identified from individual types of data. The heat maps (right) show the sample consensus obtained from pair-wise clustering. Blue-to-red gradient denotes the percentage of agreement in the clustering results in 100 clustering trials with a determined number of clusters (k=3). The color bars at the bottom represent samples belonging to clusters: RNA1-3 (A); Prot1-5 (C); and Phos1-4 (D). FIG. 6(B) shows the proportions of subtypes (see subtype legend) defined based on mRNA signatures provided by Moffitt et al., 2015, Collisson et al., 2011, Bailey et al., 2016) in RNA1-3, and uncorrelated tumors in the PDAC cohort. FIGS. 6(C) and 6(D) show global clustering results of proteome data (C) and phosphorylated proteome data (D). See legend in (A). The numbers (k) of clusters were determined to be k=5 and 4 for protein (C) and phosphorylation (D) data, respectively.

FIGS. 7(A) to 8(C) relate to association of PDAC subtypes with distinct cellular networks.

FIG. 7(A) shows cellular pathways represented by genes (S1-G to S6-G) and proteins (51-P to S6-P) defining Sub1-6. The heat map shows the enrichment significance of cellular pathways by genes or proteins defining Sub1-6. The significance is indicated as −log 10(p), where p is the p value for enrichment. FIG. 7(B) and 7(C) show a network model showing interactions between the genes and proteins involved in immune-related pathways (top of (B)) and pancreatic secretion (bottom of (B)), and RHOA signals associated with Sub5-6 (B) and Sub2-4 (C), respectively. Node colors (center and boundary) indicate whether the corresponding gene and protein were selected as signatures for Sub5-6 (green for Sub5, dark green for Sub6) and Sub2-4 (orange for Sub2, red for Sub3, dark red for Sub4). A circled P on a node indicates phosphopeptides that define the corresponding subtypes. Arrows, activation; inhibition symbols, inhibition; solid arrows, direct activation; dotted arrows, indirect activation; gray lines, protein-protein interactions. FIG. 7(D) shows the distribution of tumor cellularity in Sub1-6. The median cellularity is indicated by the red line. FIG. 7(E) shows cell counts for cultured Sub4 (SNU3608) and Sub6 (SNU3573) tumors. Data are presented as mean±SEM. ****; p<0.0001 by two-way ANOVA with Sidak's post hoc correction over the entire time range.

FIG. 8 relates to gene set enrichment analysis (GSEA) for mRNA, protein and phosphorylation signatures defining Sub1-6. FIG. 8(A) shows how mRNA and protein signatures that defined Sub6 are selected and used for GSEA, as an example. Integrative clustering (top left) shows that Sub6 was defined by RNA3, Prot5, and Phos5 clusters. Based on this information, 416 genes (rna3) defining RNA3 were selected as mRNA signatures (S6-G), and 945 proteins (prot5) and 1,030 phosphopeptides (phos4) defining Prot5 and Phos5, respectively, were selected as protein signatures (S6-P) (bottom and top right). After mapping the phosphopeptides to phosphorylated proteins and combining them with 945 proteins (prot5), the resulting proteins were used for GSEA. FIG. 8(B) shows subtype distributions of the tumors that harbored somatic mutations of TP53 or ARID1A and had altered protein abundance or phosphorylation level of the corresponding protein-encoding gene. The color in the heat maps below the bar plots indicate the subtype of patients. FIG. 8(C) shows the number of patients with or without mutations for which protein abundances or intensities of phosphopeptides are higher (positive) or lower (negative) than the median across patients. The color in the stacked bar graphs indicates the subtype of patients.

FIGS. 9(A) to 10(N) relate to the inhibition of T cell proliferation by pro-tumorigenic PMN-MDSCs.

FIGS. 9(A) and 9(B) show a comparison of tumor volume over time (A) and at end point (B) in orthotopic PDAC models grafted with cells derived from Sub4 (SNU3608) and Sub6 (SNU3573) tumors (n=10/group). FIG. 9(C) shows the numbers of immune cell subsets infiltrating SNU3608 and SNU3573 tumors (n=10/group). M-MDSC, monocyte MDSC. FIGS. 9(D) and 9(E) show representative FACS data using the indicated markers. Boxes represent PMN-MDSC. Superscript H: High, L: Low. FIGS. 9(F) and 9(G) show percentages (F) and proportions (G) of the four indicated groups of PMN-MDSCs defined based on expression levels for CXCR2 and CXCR4 (n=8/group). FIG. 9(H) shows the percentage of PMN-MDSCs showing high levels of CXCR2 and CXCR4. FIG. 9(I) shows co-culture of PMN-MDSCs and T cells isolated from orthotopic PDAC models and naive Balb/c mice, respectively. A FACS scheme for CFSE analysis for CD8+ and CD4+ is shown. FIGS. 9(J) to 9(M) show CFSE intensity distribution for CD8+ (J) and CD4+ T cells (L). The MFI of CFSE is determined (K and M; n=3-4/group). *, p<0.05; ***, p<0.001; ****, p<0.0001 by two-way ANOVA with Sidak's post hoc corrections (A, C and F) and Student's t-test (B, H, K and M).

FIG. 10(A) shows mRNA and protein expression patterns of immune cell markers across Sub1-6. The heat maps show mRNA (left) and protein (right) Z-scores of the markers in Sub1-6. In each subtype, for each marker, Z-scores were computed by auto-scaling the median expression level in the subtype using the median and standard deviation of expression levels across Sub1-6. FIG. 10(B) shows expression level distributions of representative markers for T cell (CD4 and CD8A) and neutrophil (CXCL1, CXCL8, and LCN2) in Sub1-6. In violin plots, the center line represents the median expression level of each subtype marker. FIG. 10(C) shows a schematic procedure for development of orthotropic PDAC models transplanted with cells derived from Sub4 (SNU3608) and Sub6 (SNU3573) tumors in Balb/c-nu mice. FIG. 10(D) shows representative ultrasound images of SNU3608 and SNU3573 tumors at day 8, 22, and 36. Dotted circles represent the tumors. FIG. 10(E) shows gross images of SNU3608 and SNU3573 tumors at day 42. FIG. 10(F) shows a comparison of SNU3608 and SNU3573 tumor weights measured at day 42. p=0.062 by Student's t-test. FIG. 10(G) shows an FACS gating scheme for myeloid population and chemokine receptors. Contour lines show cell density distributions; solid lines show indicated cell populations in individual plots; and red arrow heads show FACS gating flow. FIGS. 10(H) to 10(J) show percentages of indicated immune cells in SNU3608 and SNU3573 tumors measured in blood (H), spleen (I), and bone marrow (BM, J). PMN-MDSC, polymorphonuclear myeloid-derived suppressor cells; M-MDSC, monocytic myeloid-derived suppressor cells. FIGS. 10(K) to 10(N) show the numbers or percentages of four indicated PMN-MDSC groups measured in SNU3608 and SNU3573 tumors (K), as well as in blood (I), spleen (M), and BM (N) of Balb/c-nu mice carrying SNU3608 and SNU3573 tumors defined according to the expression levels of CXCR2 and CXCR4. n=3 or 4 naïve, 8 to 10 (SNU3608 or SNU3573). *, p<0.05; **, p<0.01; ****, p<0.0001 by two-way ANOVA with Sidak's post-hoc corrections.

FIG. 11 is a schematic view of MRM-MS analysis of a mixture of proteins extracted from lesion tissues of patients with pancreatic ductal adenocarcinoma and stable isotope-labeled peptides of representative genes of subtypes 1 to 6.

FIG. 12 shows MRM signal intensity and contour maps between patient samples and representative peptides of each subtype.

FIG. 13 shows a method of processing a pancreatic cancer sample using a pancreatic cancer tissue pulverization process and ultra-high pressure cycling technology.

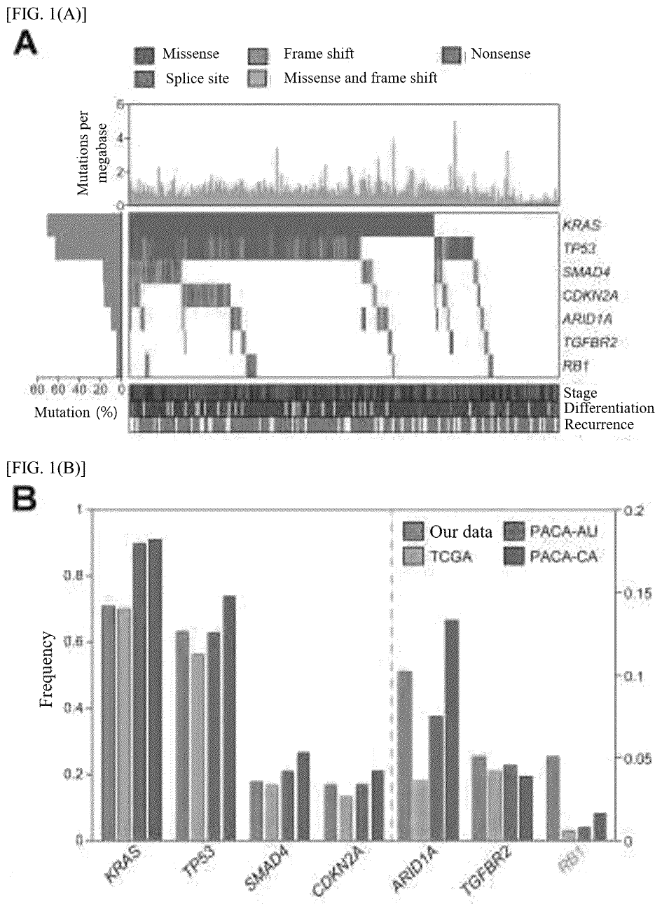

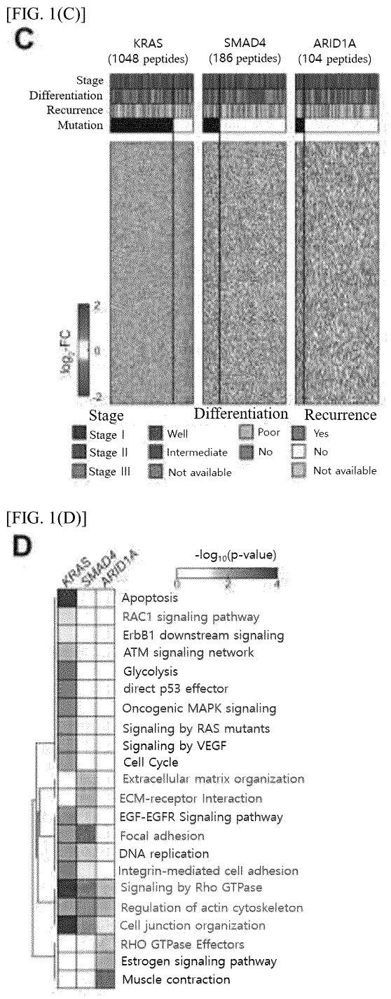

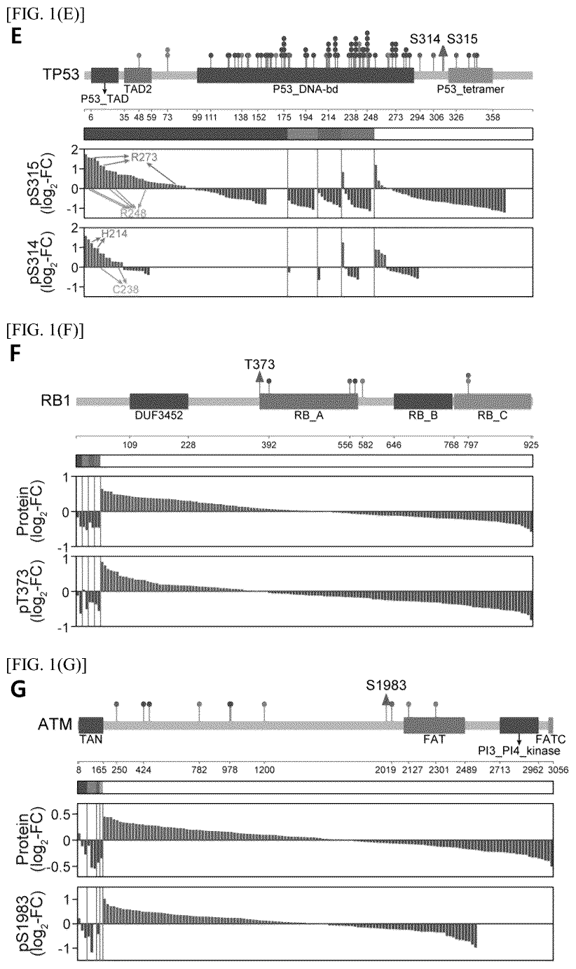

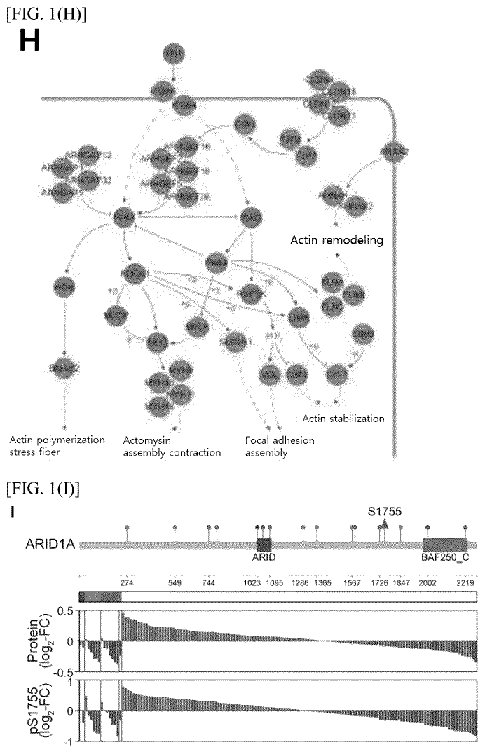

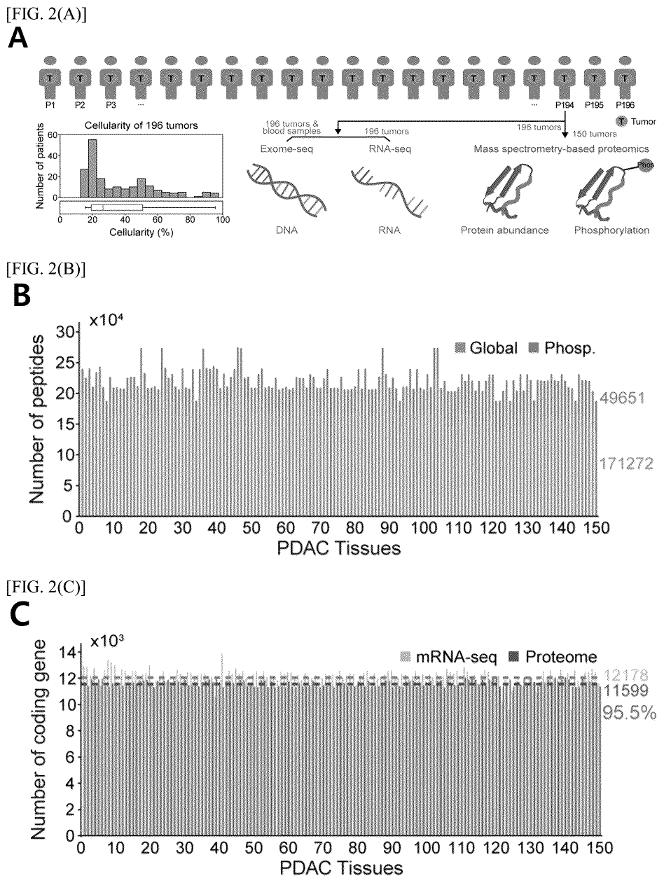

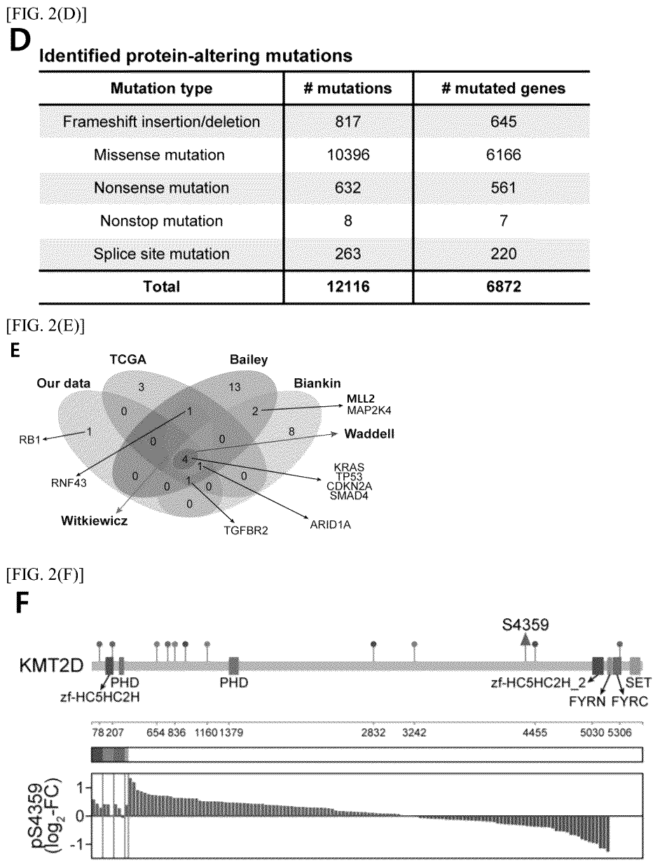

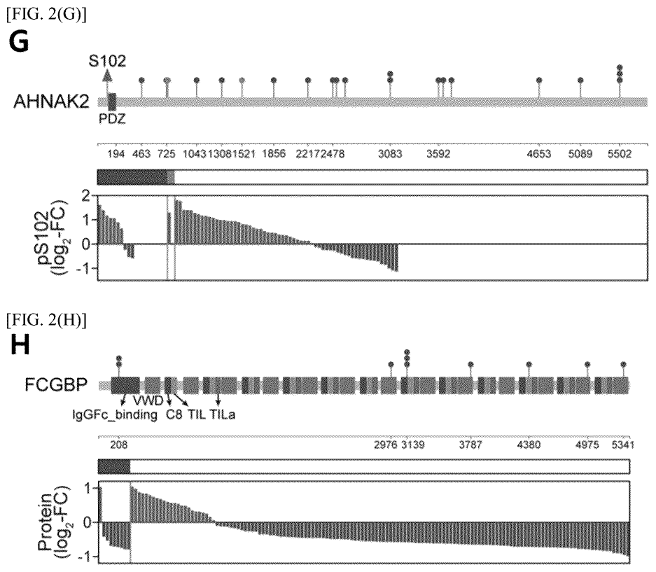

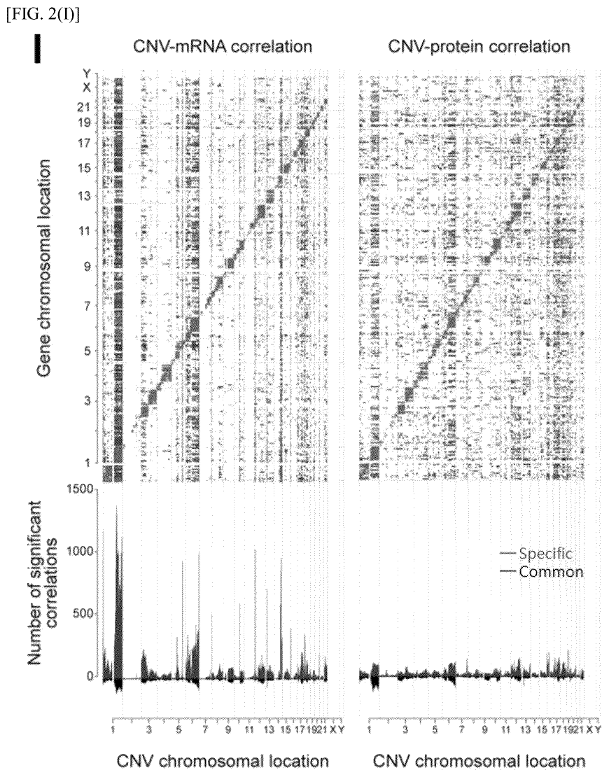

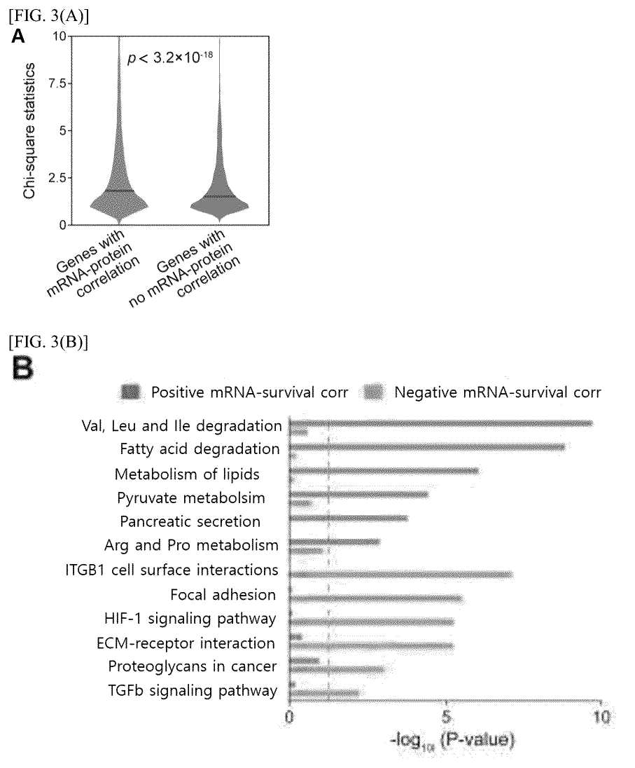

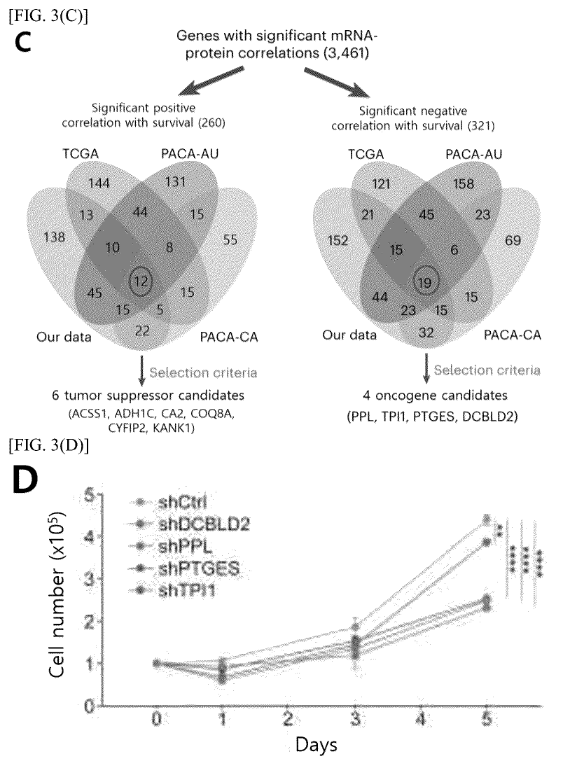

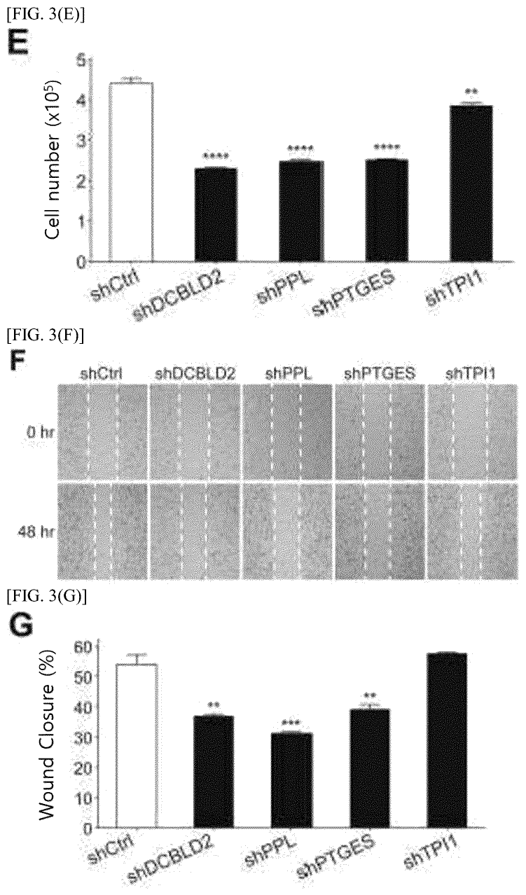

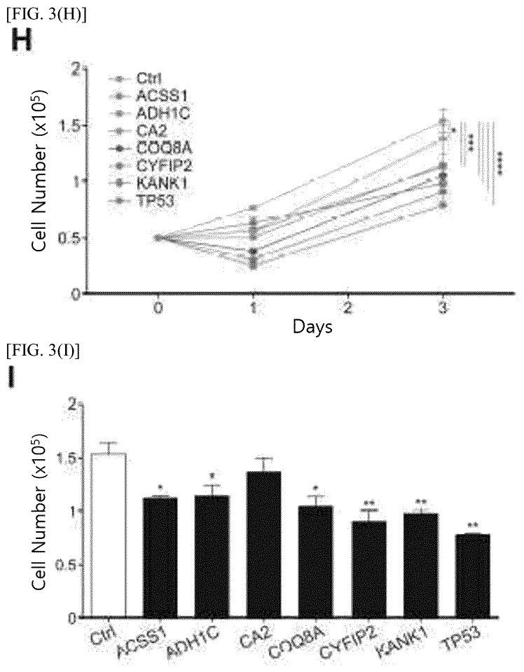

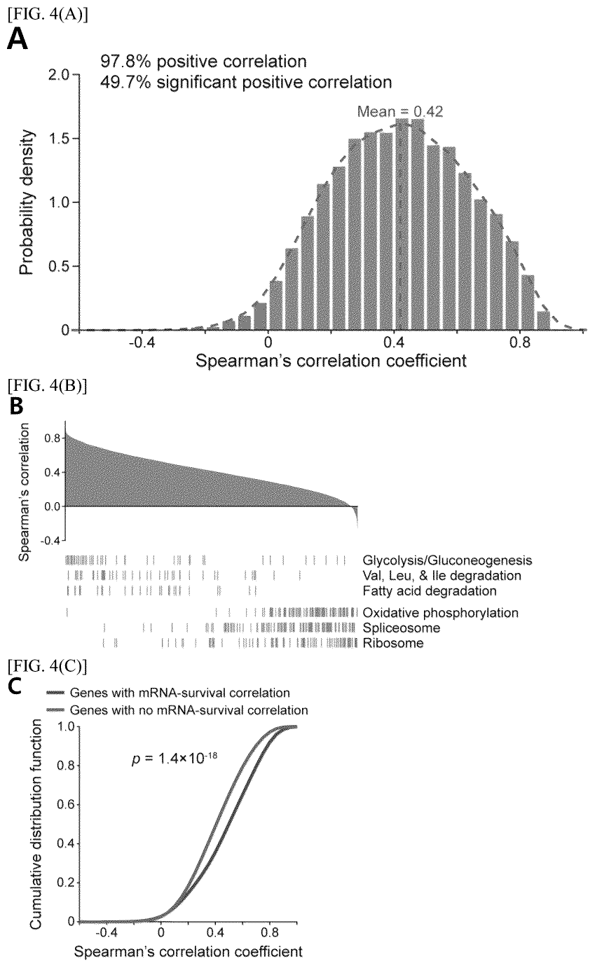

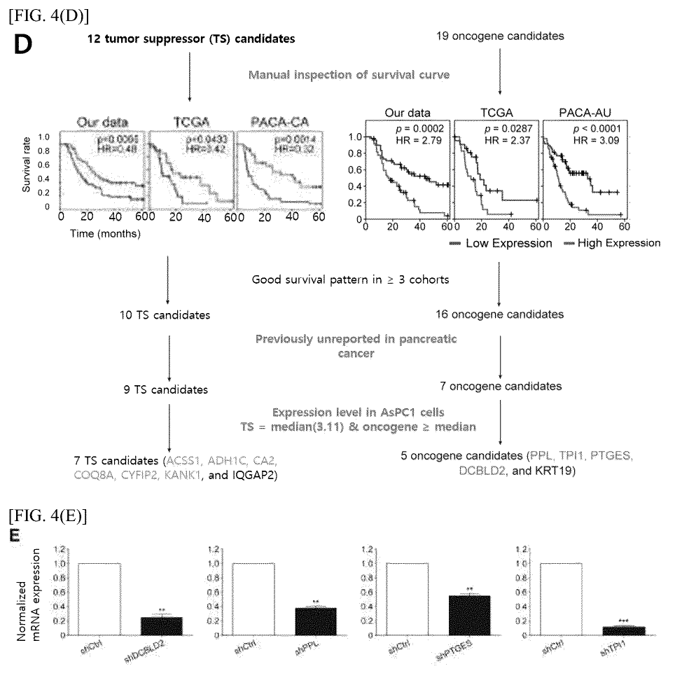

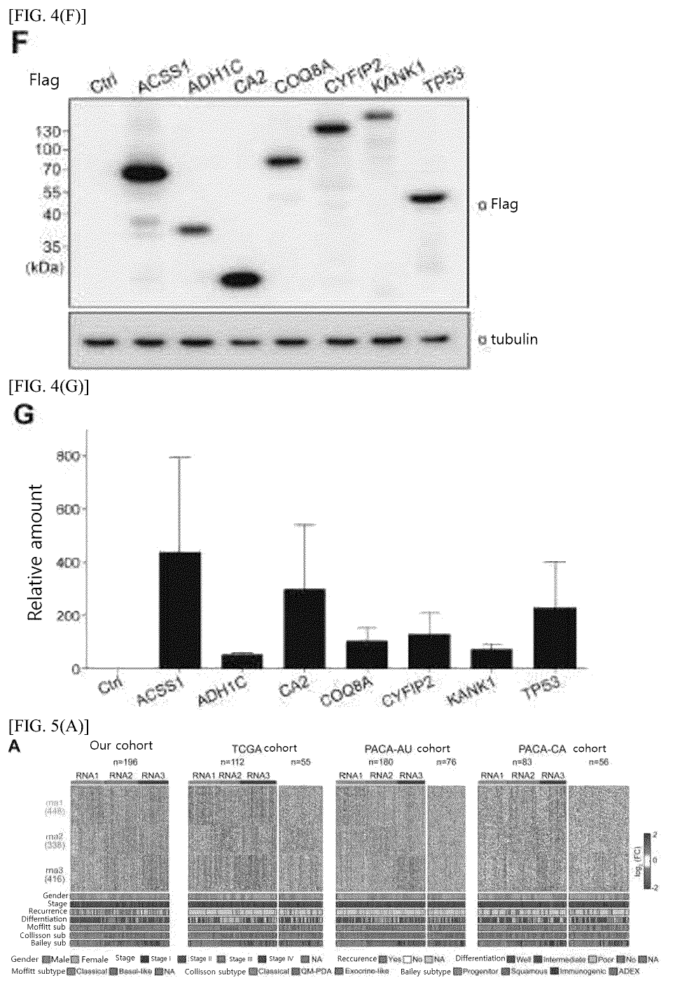

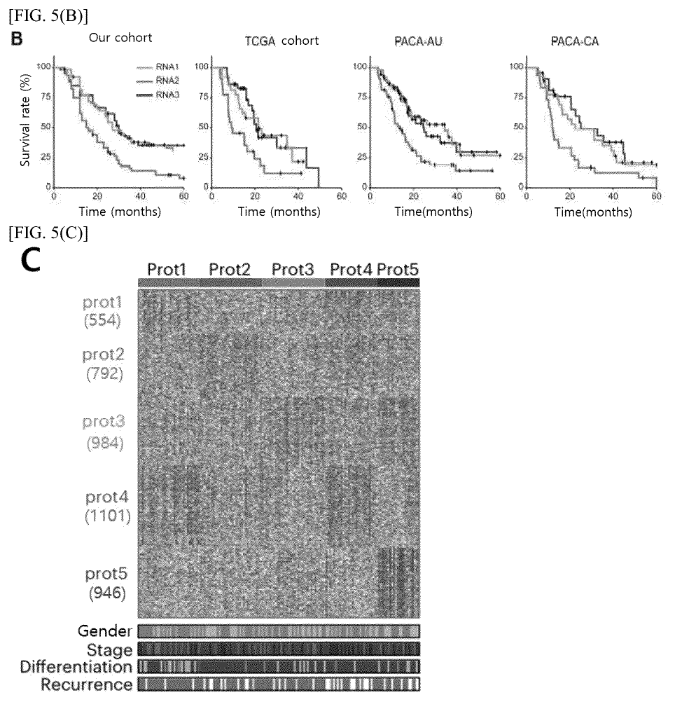

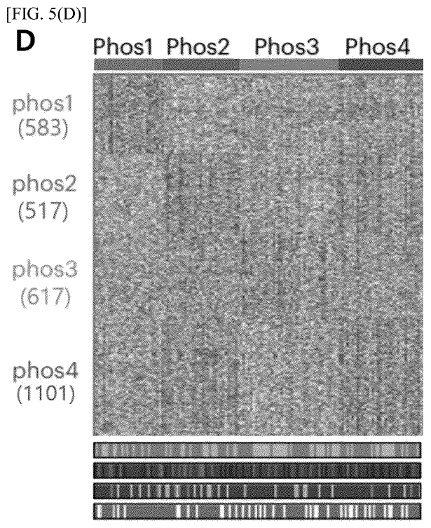

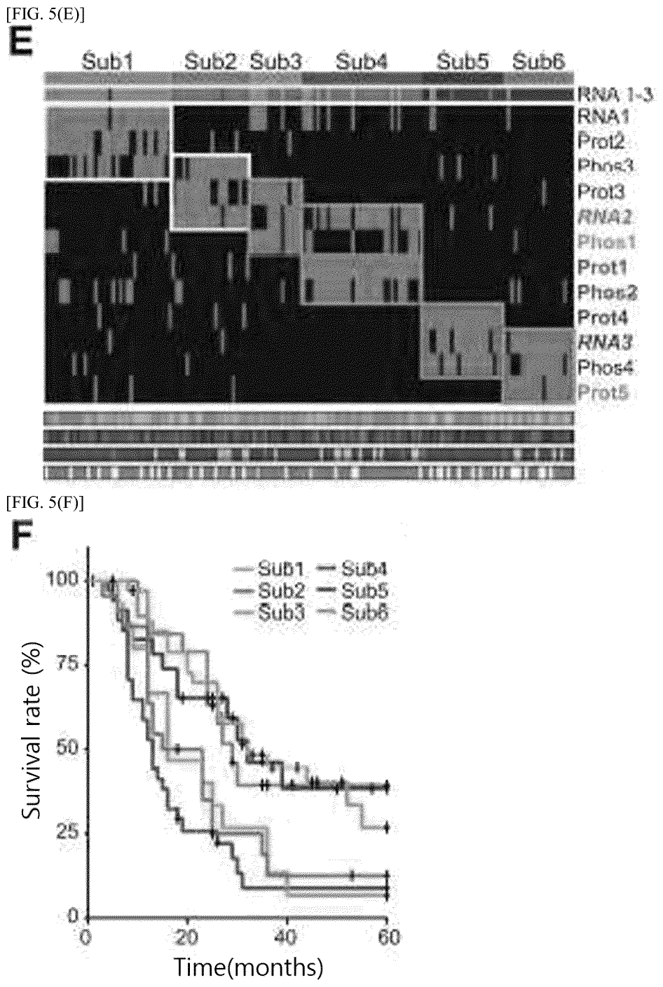

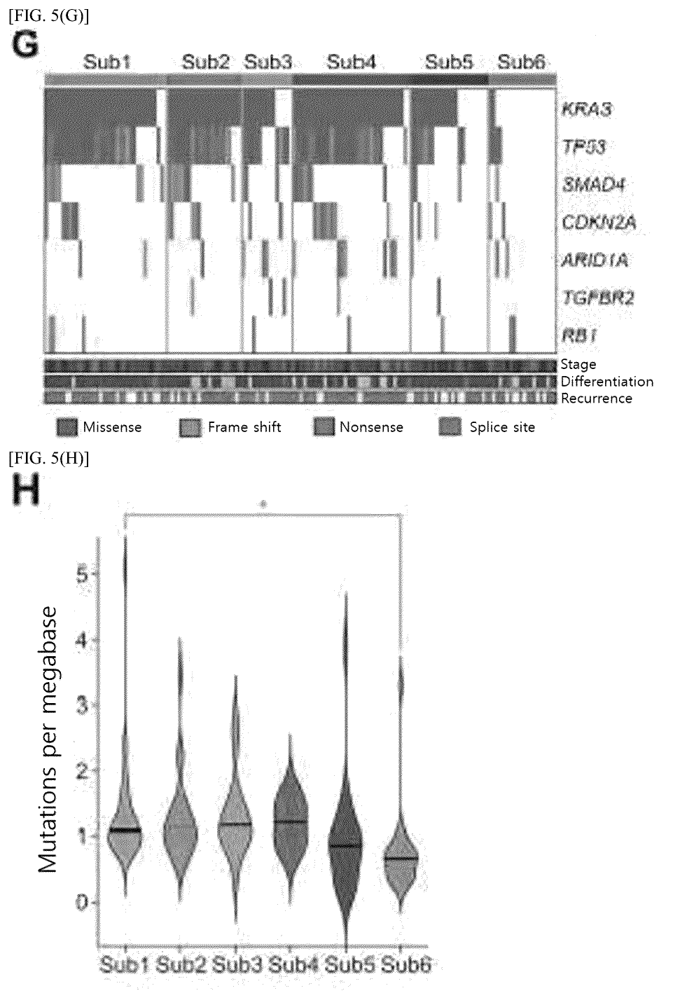

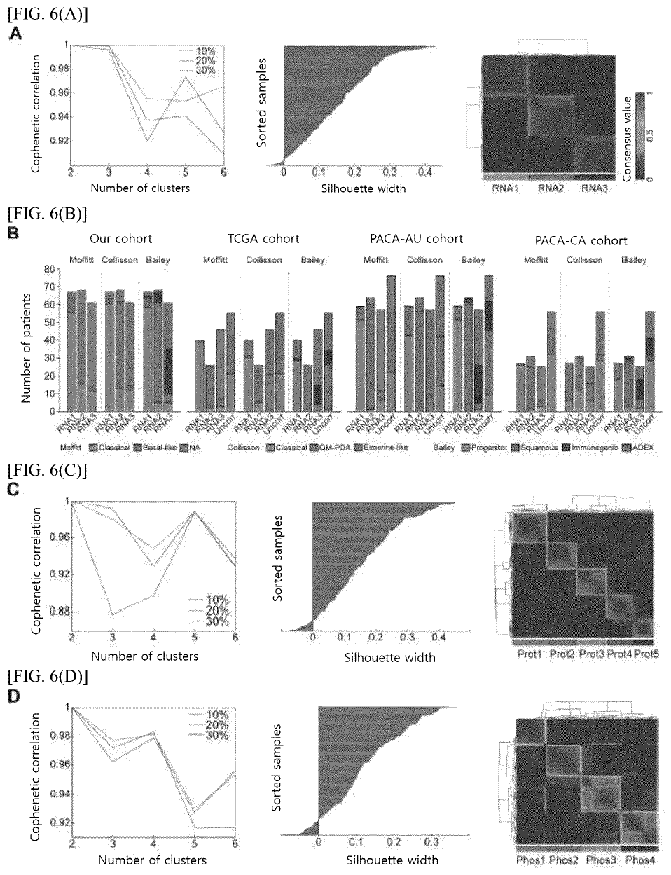

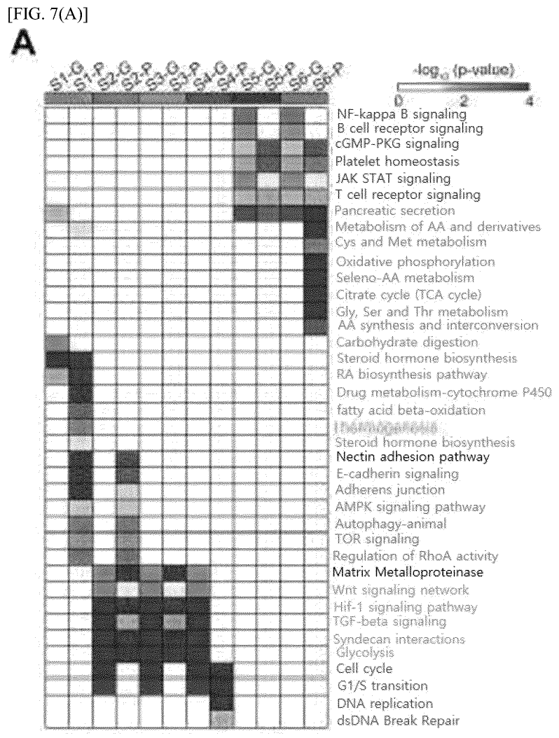

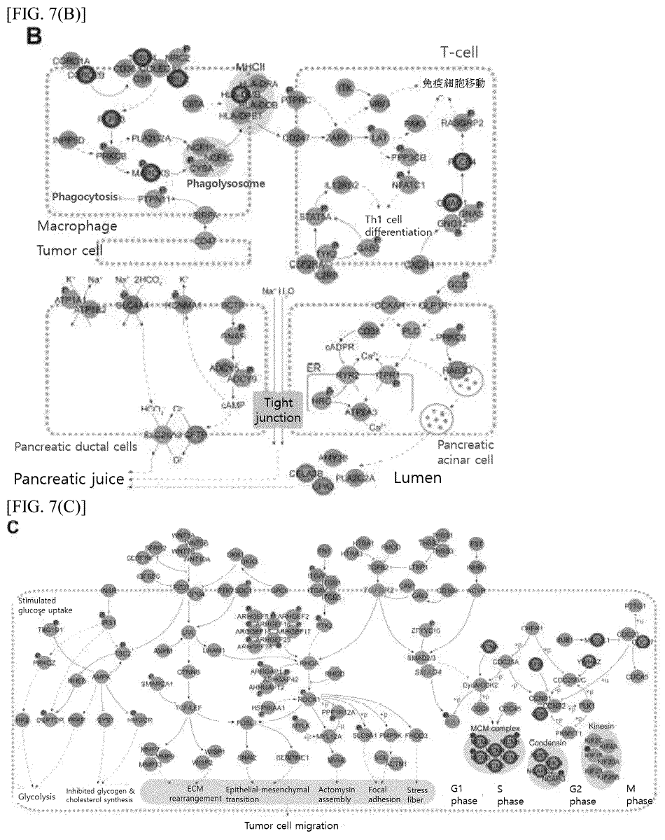

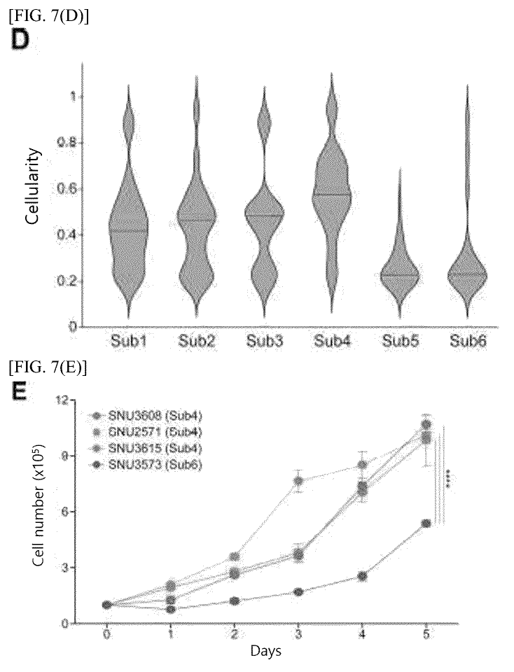

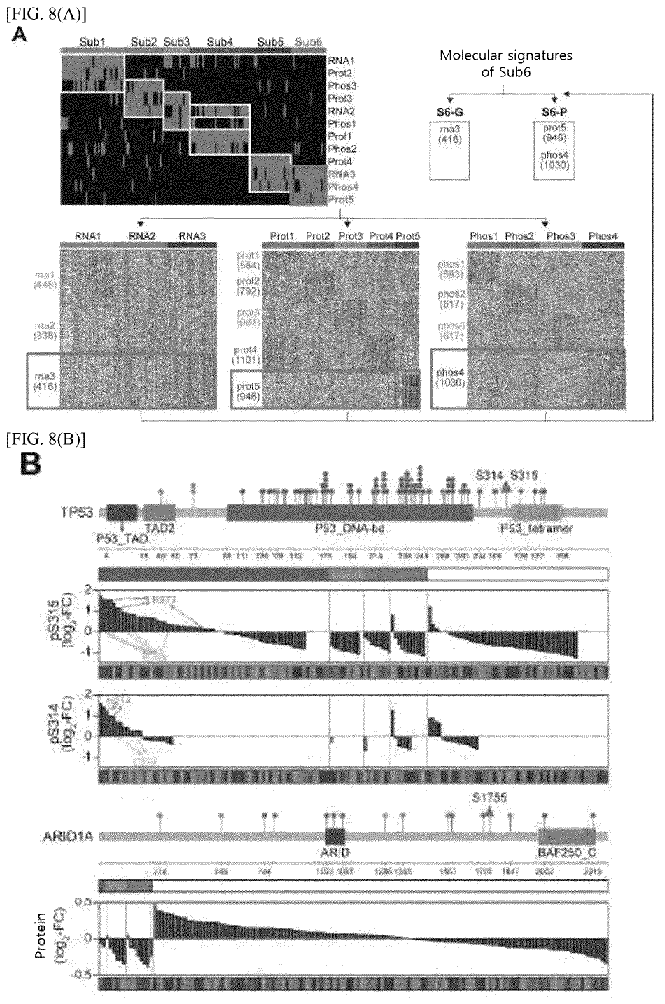

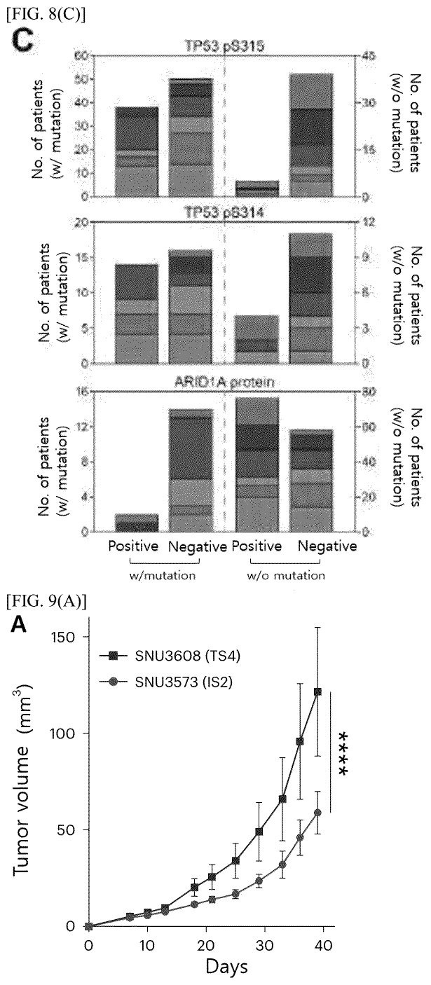

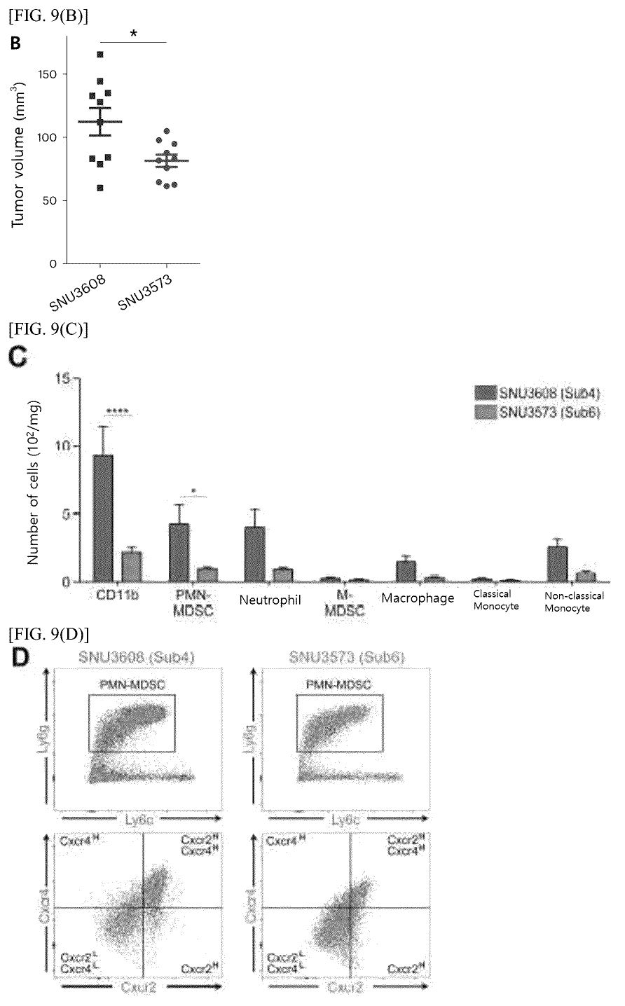

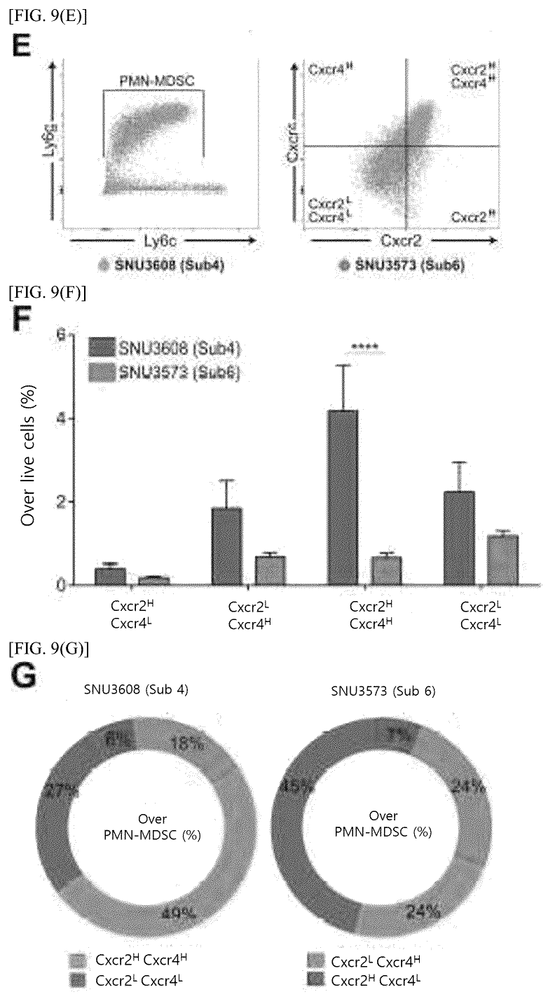

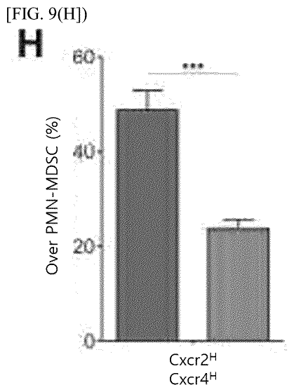

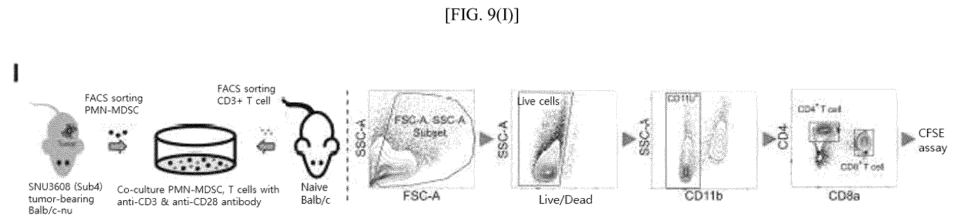

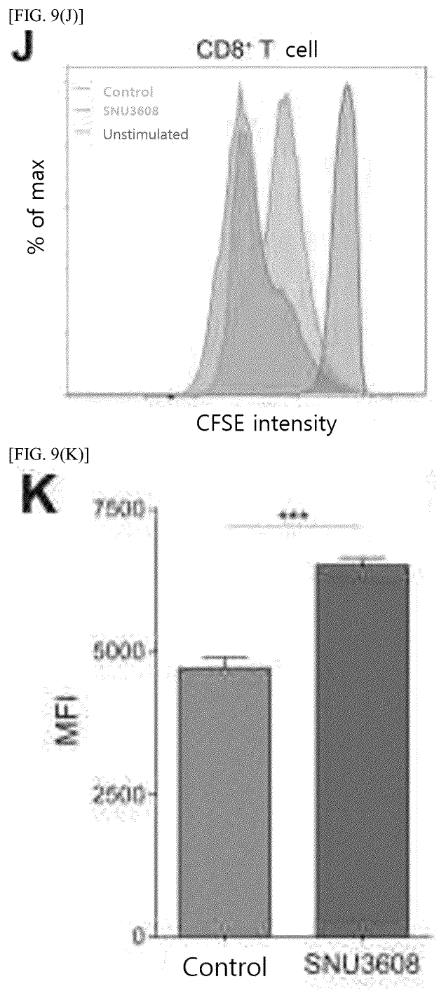

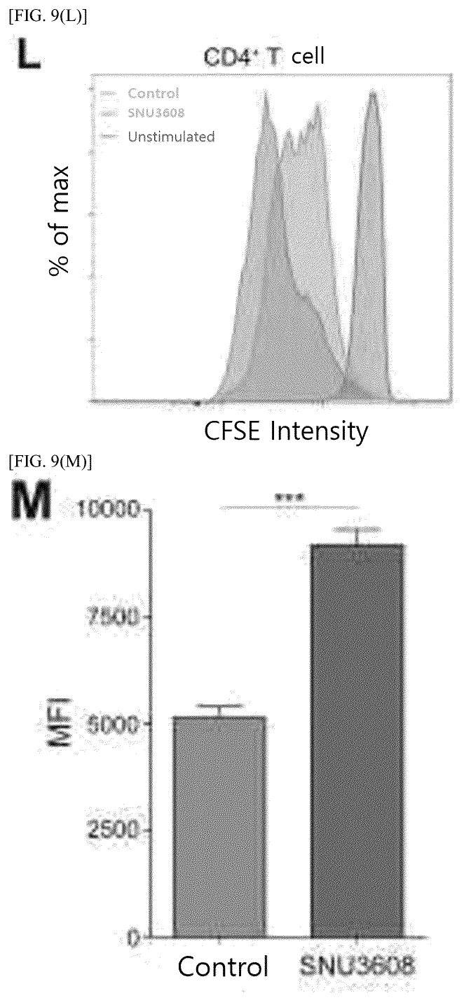

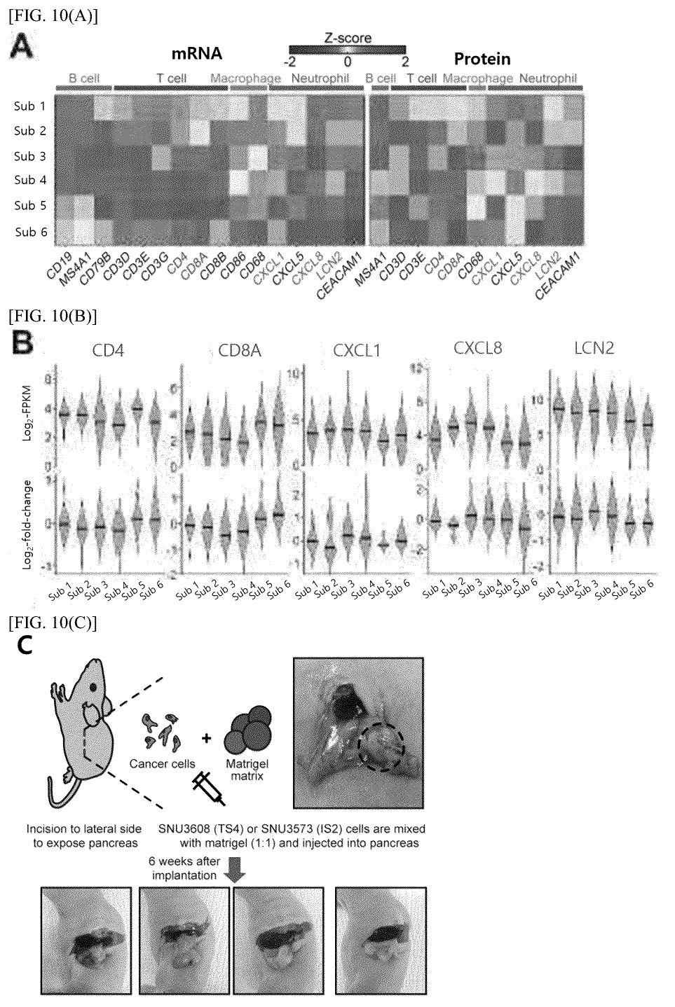

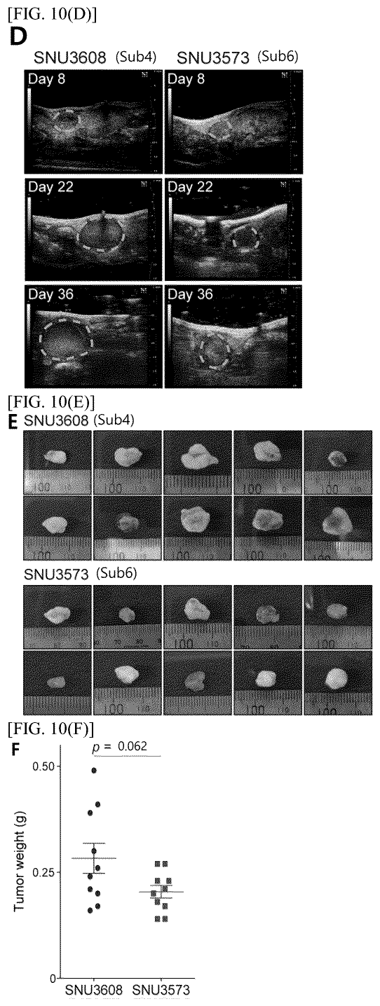

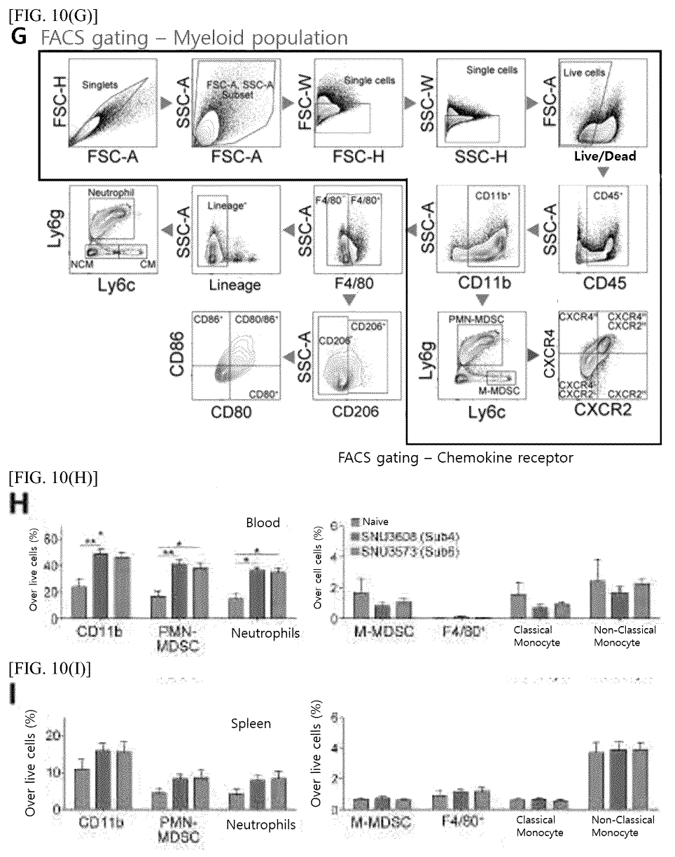

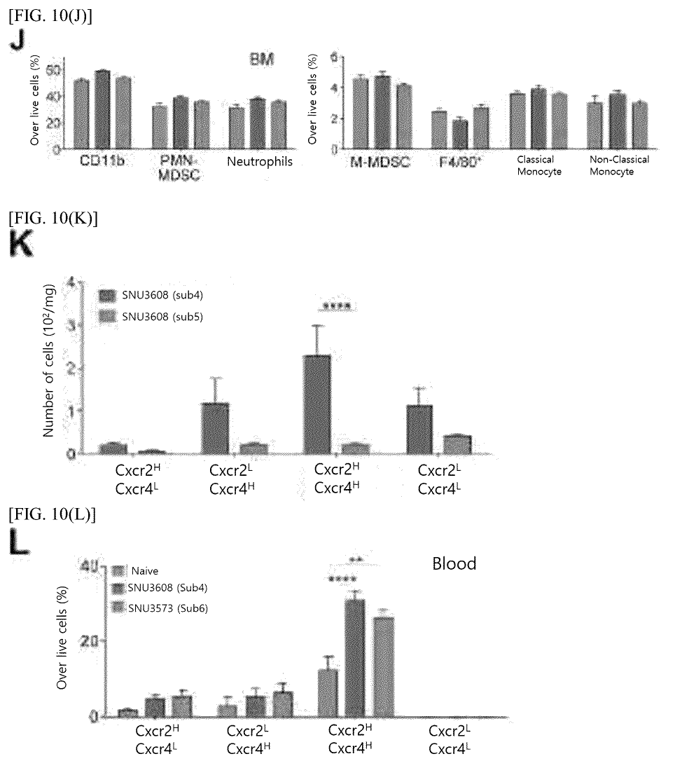

### MODE FOR INVENTION

Hereinafter, embodiments and examples of the present invention will be described in detail so that those skilled in the art can easily carry out the present invention. However, the present invention may be embodied in various different forms and is not limited to the embodiments and examples described herein.

The present invention may be variously modified and may have various forms, and specific embodiments will be described in detail in the specification. However, this is not intended to limit the present invention to specific embodiments, and it should be understood that the present invention includes all modifications, equivalents and replacements included in the spirit and technical scope of the present invention.

The terms used in the present application are used only to illustrate specific embodiments, and are not intended to limit the present invention. In the present application, it should be appreciated that terms such as “comprise(s)” or “have (has)” are intended to designate the existence of characteristics, steps, operations, components, or combinations thereof described in the specification, but are not intended to preclude the possibility of existence or addition of one or more other characteristics, steps, operations, components, or combinations thereof.

Unless otherwise defined, all terms used herein, including technical or scientific terms, have the same meanings as understood by those having ordinary knowledge in the art to which the present invention pertains. Terms such as those used in general and defined in dictionaries should be interpreted as having meanings identical to those specified in the context of related technology. Unless definitely defined in the present application, the terms should not be interpreted as having ideal or excessively formative meanings.

The present invention relates to a method and a kit for determining the subtype of pancreatic ductal adenocarcinoma.

The goal of the present invention is to identify diagnostic markers to improve pancreatic ductal adenocarcinoma (PDAC) patient stratification and improve patient management for pancreatic cancer, which is a potential therapeutic target or fatal disease.

The present invention shows that proteomic and genomic data are complementary. The availability of phosphorylation data provides information on signaling pathways with activities that correlate with somatic mutations in SMGs, suggesting the association between mutations and signaling pathways in pancreatic ductal adenocarcinoma (PDAC).

To select oncogene and tumor suppressor candidates in PDAC, mRNA-protein abundance correlation was used. In addition, to more precisely define PDAC subtypes, protein abundance and phosphorylation data were combined with mRNA abundance. GSEA and network analysis of the mRNA and protein signatures that define PDAC subtypes reveal the characteristics of the subtypes. Proteogenomic analysis through effective integration of genomic, mRNA, and proteomic data provides useful information that can help elucidate PDAC pathogenesis, stratify PDAC patients and potentially identify therapeutic targets.

The present inventors performed proteogenomic analysis of PDAC samples by combining mRNA expression data for pancreatic ductal adenocarcinoma lesion tissue samples with global proteomic data and phosphoproteomic data, thereby identifying the following representative genes of all subtypes of pancreatic ductal adenocarcinoma, the following six pancreatic ductal adenocarcinoma subtypes, and the following representative genes of each subtype:
representative genes of subtype 1 (Sub1): CLDN18, EPS8L3, CAPN5, GMDS, BCAS1, IDH1, DDAH1, SOD1, VIL1, GPX2, AOC1, LGALS4, MICU2, POF1B, MICU1, PLS1, and BDH1;
representative genes of subtype 2 (Sub2): UNC5B, PPP1R3G, IGFBP3, EDIL3,
CLSTN1, COL11A1, P4HA1, PDLIM4, ST5, FSTL1, PPP1R13L, PLTP, PDLIM7, and CALU;
representative genes of subtype 3 (Sub3): MYH9, FLNA, P4HA2, LOXL2, FN1, CD55, FLT1, ECM1, CCDC80, TSKU, HTRA1, COL12A1, SPON2, and ANGPTL2;
representative genes of subtype 4 (Sub4): PLEC, LPGAT1, NRDC, PRPF40A, CSDE1, IPO7, CDK1, HMGA1, DDX5, RASA1, ADSS, GMPS, CSE1L, PSME3, CAPRIN1, and BZW1;
representative genes of subtype 5 (Sub5): HSPB6, HSPA12A, ANXA6, VIM, UCHL1, PRPH, MAP1B, CD81, ANK2, AKAP12, ITSN1, RTN1, COL28A1, KCTD12, SPON1, SYNPO2, and EPB41L3;
representative genes of subtype 6 (Sub6): CTNND2, DTNA, REG1A, PRSS2, CPA1, CPB1, ACAT1, CPA2, PNLIPRP1, PRDX4, SNTB1, PDCD4, CTRC, FKBP11, and SEC11C;
representative genes of all subtypes (All Sub) of pancreatic ductal adenocarcinoma: KRT19, RAB27B, QSOX1, VILL, GNPAT, ABCC3, GP2, ETHE1, BPNT1, AGR2, PIGR, SRC, CTSE, JUP, RPL7, TSPAN8, SRM, VDAC1, SCP2, RPS3, AK4, RPL9, RDX, RPL3, RPL13A, RPL5, RPS9, HK2, RAB25, GNG2, RPL15, RPL37, RPS7, RPL8, RPL18A, RPL6, PABPC4, INF2, SLC25A24, MYH14, GALNT7, GOLM1, MCU, GSDMB, CYP2S1, HTATIP2, SDCBP2, SYTL2, PREB, MYO6, PKP3, SNTB2, and S100A11.

The method for determining the subtype of pancreatic ductal adenocarcinoma according to one embodiment of the present invention may comprise the following steps (1) to (4):
(1) pulverizing a pancreatic ductal adenocarcinoma lesion tissue isolated from a pancreatic ductal adenocarcinoma patient;
(2) obtaining a peptide sample for the patient by extracting and digesting proteins from the lesion tissue;
(3) measuring the expression levels of representative genes of pancreatic ductal adenocarcinoma subtypes 1 to 6 from the peptide sample for the patient, wherein the representative genes of pancreatic ductal adenocarcinoma subtypes 1 to 6 are selected from the group consisting of the following genes:
representative genes of subtype 1 (Sub1): CLDN18, EPS8L3, CAPN5, GMDS, BCAS1, IDH1, DDAH1, SOD1, VIL1, GPX2, AOC1, LGALS4, MICU2, POF1B, MICU1, PLS1, and BDH1;
representative genes of subtype 2 (Sub2): UNC5B, PPP1R3G, IGFBP3, EDIL3, CLSTN1, COL11A1, P4HA1, PDLIM4, ST5, FSTL1, PPP1R13L, PLTP, PDLIM7, and CALU;
representative genes of subtype 3 (Sub3): MYH9, FLNA, P4HA2, LOXL2, FN1, CD55, FLT1, ECM1, CCDC80, TSKU, HTRA1, COL12A1, SPON2, and ANGPTL2;
representative genes of subtype 4 (Sub4): PLEC, LPGAT1, NRDC, PRPF40A, CSDE1, IPO7, CDK1, HMGA1, DDX5, RASA1, ADSS, GMPS, CSE1L, PSME3, CAPRIN1, and BZW1;
representative genes of subtype 5 (Sub5): HSPB6, HSPA12A, ANXA6, VIM, UCHL1, PRPH, MAP1B, CD81, ANK2, AKAP12, ITSN1, RTN1, COL28A1, KCTD12, SPON1, SYNPO2, and EPB41L3; and
representative genes of subtype 6 (Sub6): CTNND2, DTNA, REG1A, PRSS2, CPA1, CPB1, ACAT1, CPA2, PNLIPRP1, PRDX4, SNTB1, PDCD4, CTRC, FKBP11, and SEC11C; and
(4) determining the subtype of the pancreatic ductal adenocarcinoma patient by comparing the expression levels of the representative genes of pancreatic ductal adenocarcinoma subtypes 1 to 6.

According to one embodiment of the present invention, the expression levels of the representative genes of subtypes 1 to 6 may be compared with the expression levels of the representative genes of all subtypes (All Sub) of pancreatic ductal adenocarcinoma. Accordingly, the reliability of subtype determination may be increased. More specifically, the expression levels of the genes most contributing to distinguishing subtypes 1 to 6 and the following all subtypes of pancreatic ductal adenocarcinoma from one another may be combined and compared.

The representative genes of all subtypes (All Sub) of pancreatic ductal adenocarcinoma may be selected from the group consisting of KRT19, RAB27B, QSOX1, VILL, GNPAT, ABCC3, GP2, ETHE1, BPNT1, AGR2, PIGR, SRC, CTSE, JUP, RPL7, TSPAN8, SRM, VDAC1, SCP2, RPS3, AK4, RPL9, RDX, RPL3, RPL13A, RPL5, RPS9, HK2, RAB25, GNG2, RPL15, RPL37, RPS7, RPL8, RPL18A, RPL6, PABPC4, INF2, SLC25A24, MYH14, GALNT7, GOLM1, MCU, GSDMB, CYP2S1, HTATIP2, SDCBP2, SYTL2, PREB, MYO6, PKP3, SNTB2, and S100A11.

According to one embodiment of the present invention, the measurement and comparison of the expression levels of the representative gene of all subtypes of pancreatic ductal adenocarcinoma and the representative genes of subtypes 1 to 6 may be performed by steps of: constructing a stable isotope-labeled peptide panel representing the representative genes of all subtype of pancreatic ductal adenocarcinoma and the genes of each subtype; mixing the patient-specific peptide sample and the stable isotope-labeled peptide panel; and determining the subtype of the pancreatic ductal adenocarcinoma patient by analyzing the mixture by quantitative mass spectrometry.

The quantitative mass spectrometry may be multiple reaction monitoring-mass spectrometry (MRM-MS), parallel reaction monitoring-mass spectrometry (PRM-MS), data independent acquisition mass spectrometry (DIA-MS), or the like, without being limited thereto.

FIG. 11 is a schematic view showing performing MRM-MS analysis of a mixture of peptides extracted from lesion tissues of pancreatic ductal adenocarcinoma patients and a panel of stable isotope-labeled peptides of representative genes of subtypes 1 to 6.

Multiple reaction monitoring/mass spectrometry (MRM-MS) using a triple quadrupole (QQQ) mass spectrometer is a method of inducing ions on a quadruple anode composed of four electrode columns and analyzing them according to the mass/charge ratio. A peptide (precursor ion, MS1) having a mass/charge specific to the selected target proteins is selected, and a fragment ion (MS2) having a characteristic mass/charge is selected from among fragments generated when the peptide collides with the second quadrupole. At this time, the pair of precursor ion/fragment ion obtained from MS1 and MS2, respectively, is referred to as the specific transition of the target protein (specific mass fingerprint of the target protein). If all these transitions are measured by multiple reaction monitoring/mass spectrometry for all target proteins (100 to 300 proteins), the relative or absolute quantities of all of the target proteins in the sample can be simultaneously analyzed within a short time through a standard material, which is a peptide of the same amino acid sequence substituted with an isotope for which quantitative information is known. According to this principle, MRM-MS is capable of selectively detecting and quantifying only a target analyte with high sensitivity, and the cost required for analysis may be reduced.

Currently, the representative method most frequently used for protein quantitative analysis is a method that relies on antibodies, such as ELISA assay, which is costly and time-consuming in the process of finding new antibodies and optimizing the analysis process.

According to one embodiment of the present invention, the MRM-MS analysis may be performed by comparing the signal intensities of the patient-derived peptides with those of the representative peptides of each subtype, and the signal intensity ratio may be expressed as signal-intensity contour map for each peptide with the peptide and the peptide elution time as two axes.

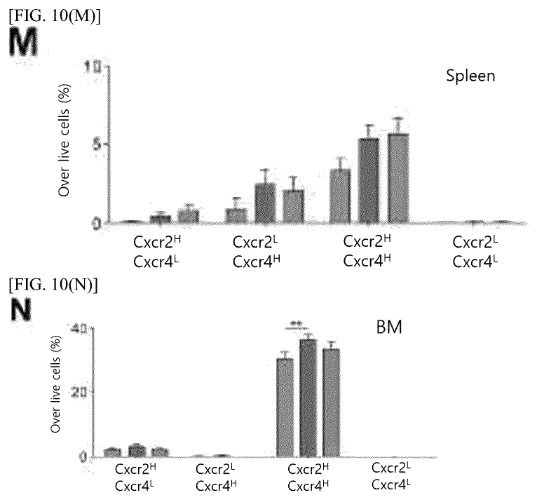

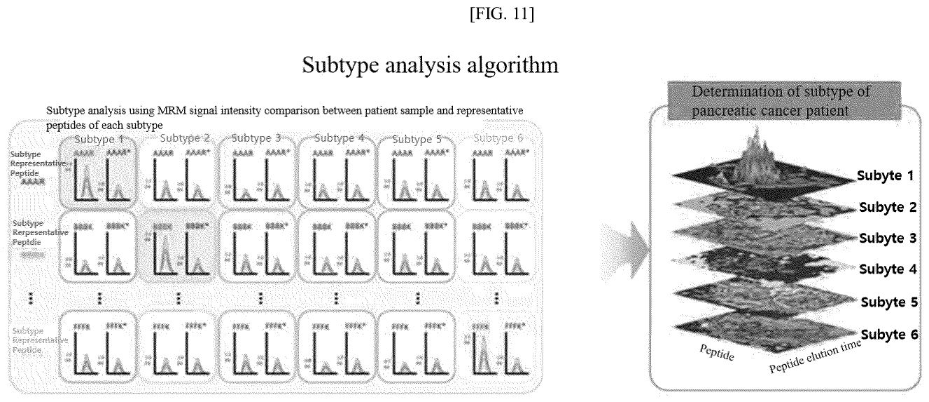

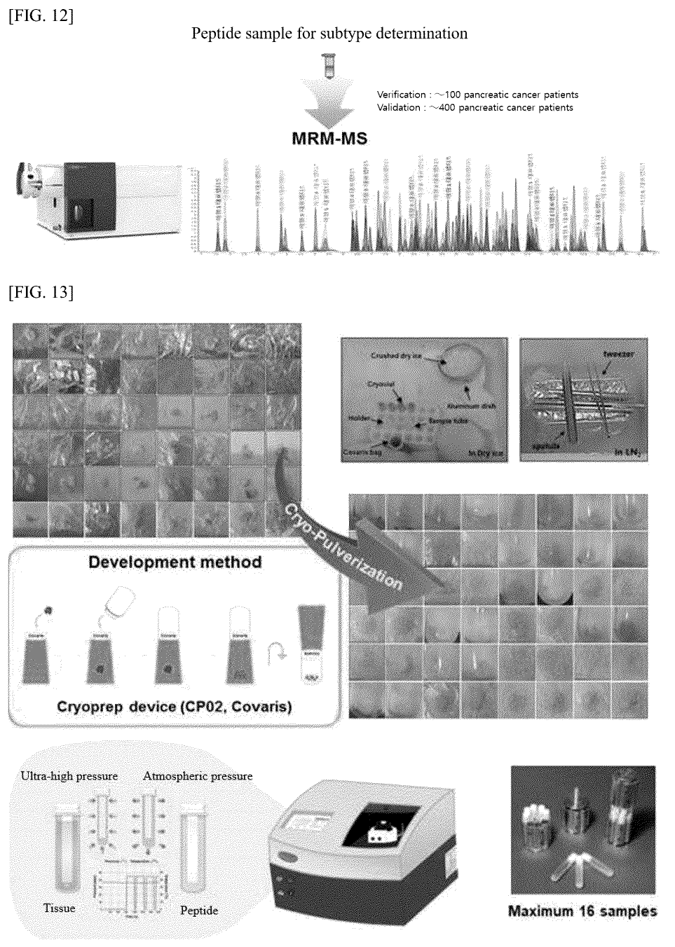

FIG. 12 is an MRM signal intensity and contour map between a patient sample and the representative peptides of each subtype.

This intensity contour map for the representative peptide for each subtype may be used for pattern comparison with an intensity contour map for the representative peptide for each subtype, obtained from endoscopic tissue of a pancreatic cancer patient visiting a hospital for diagnosis, thereby determining the subtype of the patient.

According to one embodiment of the present invention, pulverizing the pancreatic ductal adenocarcinoma lesion tissue in step (1) may be performed by cryogenic pulverization. Fine tissue powder may be obtained by the cryogenic pulverization.

The cryogenic pulverization is an optimal tissue sample processing technique for minimizing loss of tumor tissue. The cryogenic pulverization may be performed at liquid nitrogen temperature (−196° C.), without being limited thereto.

In order to perform subtype determination on large-scale pancreatic cancer patients, patient tissue-derived peptide samples to be mixed with representative peptides of each subtype should be rapidly obtained. The first process for obtaining a peptide from tissue is a tissue homogenization process, which can minimize tissue degeneration by treating the tissue within 1 minute in a cryogenic state.

Cryogenic pulverization is a method optimized even for a very small amount of a pancreatic cancer patient sample because there is no process of exposing the tissue to the outside during the process of pulverizing the tissue into a powder state, and thus no loss of the sample occurs.

According to one embodiment of the present invention, step (2) of obtaining the peptide sample by extracting and digesting the proteins may be performed by pressure cycling technology.

The pressure cycling technology is one in which ultra-high pressure (45,000 psi) and low pressure (about 15 psi) are alternately applied to the pulverized pancreatic cancer tissue sample, thereby extracting and digesting proteins more effectively. According to this technology, the time to obtain the peptide from the tissue may be 3 hours or less. This is very fast compared to an existing method, which takes 30 hours, and is a high-efficiency technique which may be applied to 16 tissue samples at the same time. Therefore, it is possible to perform subtype determination for a large number of patients.

FIG. 13 shows a method of processing a pancreatic cancer sample using a pancreatic cancer tissue pulverization process and ultra-high pressure cycling technology.

According to one embodiment of the present invention, subtypes 2 to 4 may have invasive characteristics, and subtypes 5 and 6 may have immunogenicity.

In addition, subtype 4 may have invasive characteristics and proliferative properties, and may have low T-cell proliferation.

According to one embodiment of the present invention, subtypes 2 to 4 may be associated with epithelial-to-mesenchymal transition (EMT)-related pathways.

In addition, subtype 5 and subtype 6 may be associated with immune-related pathways.

Subtype 1 may be involved in carbohydrate/lipid metabolism.

The PDAC subtypes include not only mRNA/protein signatures and cellular pathways for each subtype, but also anti-inflammatory immune cell profiles. PDAC patients may be further stratified according to prognosis by being classified as subtypes 2-4 (poor-prognosis subtypes) or subtypes 1, 5 and 6 (good-prognosis subtypes) based on the mRNA and protein signatures of their tumors.

Another embodiment of the present invention relates to a kit capable of determining subtypes of pancreatic ductal adenocarcinoma. The kit for determining the subtype of pancreatic ductal adenocarcinoma according to one embodiment of the present invention may comprise agents for measuring the expression levels of representative genes of pancreatic ductal adenocarcinoma subtypes 1 to 6, wherein the representative genes of pancreatic ductal adenocarcinoma subtypes 1 to 6 may be selected from the group consisting of the following genes:
representative genes of subtype 1 (Sub1): CLDN18, EPS8L3, CAPN5, GMDS, BCAS1, IDH1, DDAH1, SOD1, VIL1, GPX2, AOC1, LGALS4, MICU2, POF1B, MICU1, PLS1, and BDH1;
representative genes of subtype 2 (Sub2): UNC5B, PPP1R3G, IGFBP3, EDIL3, CLSTN1, COL11A1, P4HA1, PDLIM4, ST5, FSTL1, PPP1R13L, PLTP, PDLIM7, and CALU;
representative genes of subtype 3 (Sub3): MYH9, FLNA, P4HA2, LOXL2, FN1, CD55, FLT1, ECM1, CCDC80, TSKU, HTRA1, COL12A1, SPON2, and ANGPTL2;
representative genes of subtype 4 (Sub4): PLEC, LPGAT1, NRDC, PRPF40A, CSDE1, IPO7, CDK1, HMGA1, DDX5, RASA1, ADSS, GMPS, CSE1L, PSME3, CAPRIN1, and BZW1;
representative genes of subtype 5 (Sub5): HSPB6, HSPA12A, ANXA6, VIM, UCHL1, PRPH, MAP1B, CD81, ANK2, AKAP12, ITSN1, RTN1, COL28A1, KCTD12, SPON1, SYNPO2, and EPB41L3; and
representative genes of subtype 6 (Sub6): CTNND2, DTNA, REG1A, PRSS2, CPA1, CPB1, ACAT1, CPA2, PNLIPRP1, PRDX4, SNTB1, PDCD4, CTRC, FKBP11, and SEC11C.

According to one embodiment of the present invention, the kit for determining the subtype of pancreatic ductal adenocarcinoma may comprise agents for measuring the expression levels of representative genes of all subtypes (All Sub) of pancreatic ductal adenocarcinoma, wherein the expression levels of representative genes of subtypes 1 to 6 may be compared with the expression levels of the representative genes of all subtypes of pancreatic ductal adenocarcinoma.

The representative genes of all subtypes (All Sub) of pancreatic ductal adenocarcinoma may be selected from the group consisting of KRT19, RAB27B, QSOX1, VILL, GNPAT, ABCC3, GP2, ETHE1, BPNT1, AGR2, PIGR, SRC, CTSE, JUP, RPL7, TSPAN8, SRM, VDAC1, SCP2, RPS3, AK4, RPL9, RDX, RPL3, RPL13A, RPL5, RPS9, HK2, RAB25, GNG2, RPL15, RPL37, RPS7, RPL8, RPL18A, RPL6, PABPC4, INF2, SLC25A24, MYH14, GALNT7, GOLM1, MCU, GSDMB, CYP2S1, HTATIP2, SDCBP2, SYTL2, PREB, MYO6, PKP3, SNTB2, and S100A11.

According to one embodiment of the present invention, the agents for measuring the expression levels of the representative genes of all subtypes of pancreatic ductal adenocarcinoma and the representative genes of subtypes 1 to 6 may comprise a stable isotope-labeled peptide panel representing the representative genes of all subtype of pancreatic ductal adenocarcinoma and the genes of each subtype.

Another embodiment of the present invention provides a method for predicting the prognosis of a pancreatic ductal adenocarcinoma patient, the method comprising the following steps (1) to (5):
(1) pulverizing a pancreatic ductal adenocarcinoma lesion tissue isolated from a pancreatic ductal adenocarcinoma patient;
(2) obtaining a peptide sample for the patient by extracting and digesting proteins from the lesion tissue;
(3) measuring the expression levels of representative genes of pancreatic ductal adenocarcinoma subtypes 1 to 6 from the peptide sample for the patient, wherein the representative genes of pancreatic ductal adenocarcinoma subtypes 1 to 6 are selected from the group consisting of the following genes:
representative genes of subtype 1 (Sub1): CLDN18, EPS8L3, CAPN5, GMDS, BCAS1, IDH1, DDAH1, SOD1, VIL1, GPX2, AOC1, LGALS4, MICU2, POF1B, MICU1, PLS1, and BDH1;
representative genes of subtype 2 (Sub2): UNC5B, PPP1R3G, IGFBP3, EDIL3, CLSTN1, COL11A1, P4HA1, PDLIM4, ST5, FSTL1, PPP1R13L, PLTP, PDLIM7, and CALU;
representative genes of subtype 3 (Sub3): MYH9, FLNA, P4HA2, LOXL2, FN1, CD55, FLT1, ECM1, CCDC80, TSKU, HTRA1, COL12A1, SPON2, and ANGPTL2;
representative genes of subtype 4 (Sub4): PLEC, LPGAT1, NRDC, PRPF40A, CSDE1, IPO7, CDK1, HMGA1, DDX5, RASA1, ADSS, GMPS, CSE1L, PSME3, CAPRIN1, and BZW1;
representative genes of subtype 5 (Sub5): HSPB6, HSPA12A, ANXA6, VIM, UCHL1, PRPH, MAP1B, CD81, ANK2, AKAP12, ITSN1, RTN1, COL28A1, KCTD12, SPON1, SYNPO2, and EPB41L3; and
representative genes of subtype 6 (Sub6): CTNND2, DTNA, REG1A, PRSS2, CPA1, CPB1, ACAT1, CPA2, PNLIPRP1, PRDX4, SNTB1, PDCD4, CTRC, FKBP11, and SEC11C;
(4) determining the subtype of the pancreatic ductal adenocarcinoma patient by comparing the expression levels of the representative genes of pancreatic ductal adenocarcinoma subtypes 1 to 6 to; and
(5) predicting prognosis of the patient based on the determined subtype.

According to one embodiment of the present invention, the expression levels of the representative genes of subtypes 1 to 6 may be compared with the expression levels of representative genes of all subtypes (All Sub) of pancreatic ductal adenocarcinoma.

The representative genes of all subtypes (All Sub) of pancreatic ductal adenocarcinoma may be selected from the group consisting of KRT19, RAB27B, QSOX1, VILL, GNPAT, ABCC3, GP2, ETHE1, BPNT1, AGR2, PIGR, SRC, CTSE, JUP, RPL7, TSPAN8, SRM, VDAC1, SCP2, RPS3, AK4, RPL9, RDX, RPL3, RPL13A, RPL5, RPS9, HK2, RAB25, GNG2, RPL15, RPL37, RPS7, RPL8, RPL18A, RPL6, PABPC4, INF2, SLC25A24, MYH14, GALNT7, GOLM1, MCU, GSDMB, CYP2S1, HTATIP2, SDCBP2, SYTL2, PREB, MYO6, PKP3, SNTB2, and S100A11.

According to one embodiment of the present invention, subtypes 2 to 4 may be predicted to have a poor prognosis compared to subtypes 1, 5, and 6.

In addition, treatment strategies may be employed based on subtypes and related pathways and/or immune cell profiles. For example, Sub4 exhibits high invasive activity and increased PMN-MDSC contributing to tumor cell proliferation by reducing T cell activity. This pattern suggests that both invasiveness and PMN-MDSCs should be addressed by targeting invasion-associated RHOA and/or TGFB signaling and pro-tumorigenic PMN-MDSCs at once upon treatment of Sub4 tumors. Interestingly, although the PDAC cohort does not include acinar cell carcinoma, Sub6 has low cellularity and has some endocrine characteristics. In low-cellularity tumors, these characteristics are suggested to occur due to dedifferentiation of ductal cells (Martens et al., 2019), large numbers of stromal cells (Bailey et al., 2016), or acinar cell contamination (Puleo et al., 2018). Proteogenomic signatures are applied to low-cellularity tumors classified as Sub6 with endocrine characteristics. However, whether they are also applicable to acinar cell carcinoma will have to be examined in large cohorts.

Numerous immune checkpoint molecules have been reported (Kalbasi and Ribas, 2020; Wei et al., 2018). The mRNA expression levels of CEACAM1, PVR and PVRL2 were higher in Sub2-4 than in Sub5-6, but the levels of CD48, IGSF11, CD96, CD244 and BTLA were higher in Sub5-6. In addition, CEACAM1, HMGB1 and CD274 showed the highest mRNA expression levels in Sub4 across all subtypes. Consistent with the mRNA data, higher levels of the proteins CEACAM1 and PVR were detected in Sub1-4 than Sub5-6, and the highest protein level of CD274 was detected in Sub4. CEACAM1, PVR and CD274 inhibit the activity of T cells, and/or natural killer (NK) cells (Qin et al., 2019). This type of immune suppression is observed in various cancers including PDAC (Dong et al., 2002; Feig et al., 2013; Nishiwada et al., 2015) Immune checkpoints identified in Sub5-6 are not detected by proteomic analysis. In addition, PMN-MDSCs, mainly pro-tumorigenic neutrophils, infiltrated Sub4 tumors at high levels. PMN-MDSC-mediated immune suppression was reported in lung cancer (Huang et al., 2013), colon cancer (Jung et al., 2017a; Jung et al., 2017b), breast cancer (Alizadeh et al., 2014), head cancer and neck cancer (Brandau et al., 2011), kidney cancer (Rodriguez et al., 2009), and stomach cancer (Wang et al., 2013), as well as in PDAC (Porembka et al., 2012). According to the human blood atlas (Uhlen et al., 2019), CEACAM1, PVR and CD274 are expressed at high levels in PMN-MDSCs, suggesting a potential association of PMN-MDSCs with immune checkpoints. How these proteins are associated with anti-tumor immunodeficiency in Sub4 tumors can be investigated through detailed functional studies in the future.

Hereinafter, the present invention will be described in more detail with reference to examples. However, these examples are intended to illustrate one or more specific examples, and the scope of the present invention is not limited to these examples.

### Examples

To define PDAC patient subtypes based on proteogenomic analysis, the present inventors first clustered patient's tumor samples using mRNA expression data, global proteomic data, and phosphoproteomic data, thereby identifying 3 (RNA1-3), 5 (Prot1-5), and 5 (Phos1-5) patient clusters, respectively. In addition, in order to understand the characteristics of each patient cluster, the signature genes (rna1-3), proteins (prot1-5) and phosphopeptides (phos1-5), which show significantly higher expression in patient samples of each cluster than in the other patient samples, were selected through statistical comparative analysis. Finally, 6 subtypes (Sub1-6) were identified by performing integrated clustering of 150 patient samples.

In order to determine the cellular processes associated with each identified subtype, the present inventors first selected signature genes and proteins corresponding to each subtype. Then, the corresponding cellular processes were identified through functional enrichment analysis for the corresponding genes and proteins. Thereby, it was confirmed that Sub2-4 commonly had high expression of epithelial-to-mesenchymal transition (EMT)-related genes, and among them, Sub2-3 had high expression of the same EMT-related proteins, whereas Sub4 had high expression of cell cycle-related proteins. In addition, it was confirmed that Sub5-6 commonly had high expression of immune-related genes, and among them, Sub5 had high expression of the same immune-related proteins, whereas Sub6 had high expression of exocrine-related proteins. Lastly, it was confirmed that Sub1 had high expression of genes and proteins related to carbohydrate/lipid metabolism, a feature of the classical progenitor PDAC subtype.

In order to identify subtype's representative peptides for classifying these six patient subtypes, the present inventors performed partial least squares (PLS) analysis for the previously selected signature proteins (prot1-5) and phosphopeptides (phos1-5). For the signature proteins, PLS analysis was performed after conversion to sibling peptides corresponding to each protein. Through PLS analysis, using the log2-fold-change value of peptides in the 150 patients, the present inventors created a model that predicts whether the 150 patients belong to specific subtypes (Sub1-6) or simultaneously predicts all subtypes of the 150 patients. In addition, the degree of contribution of individual peptides to patient subtype prediction was quantified as variable importance in projection (VIP) value.

In order to identify representative phosphopeptides of each of the six subtypes, the present inventors selected phosphopeptides that (1) were identified as signatures in the corresponding subtype, 2) had a VIP value greater than 1.5, 3) had a VIP value greater than the VIP value in the other subtypes, and 4) were detected in 80% or more of the patients. For representative phosphopeptides predicting all subtypes, the present inventors selected phosphopeptides that (1) had a VIP value greater than 1.5 and 2) were detected in 80% or more of all patients. Then, among these peptides, the present inventors selected peptides containing only one phosphorylation and suitable for use in MRM-MS analysis (considering the length of the peptide, whether there is a signal peptide, whether there is missed cleavage, etc.), thereby identifying 16 phosphopeptides. Next, in order to identify representative global peptides of each of the six subtypes, the present inventors selected proteins that 1) were identified as signatures in the corresponding subtype and 2) were detected in 80% or more of the patients. Among the sibling peptides of the selected proteins, the present inventors selected peptides that 1) had a VIP value greater than 1.15 in the corresponding subtype, 2) had a VIP value greater than the VIP value in the other subtypes, and 3) were detected in 80% or more of the patients. For representative global peptides predicting all subtypes, among the sibling peptides of proteins detected in 80% or more of all patients, the present inventors selected peptides that 1) had a VIP value greater than 1.15, and 2) were detected in 80% or more of all patients. Next, among them, no more than 2 peptides suitable for use in MRM-MS analysis were selected per each signature protein, and 132 global peptides were finally identified.

Through the above-described process, the final 150 subtype's representative peptides were identified, including the 16 phosphopeptides, the 132 finally identified global peptides, and two KRAS mutant protein peptides showing expression differences between the subtypes.

Representative genes of subtype 1 (Sub1): CLDN18, EPS8L3, CAPN5, GMDS, BCAS1, IDH1, DDAH1, SOD1, VIL1, GPX2, AOC1, LGALS4, MICU2, POF1B, MICU1, PLS1, and BDH1.

Representative genes of subtype 2 (Sub2): UNC5B, PPP1R3G, IGFBP3, EDIL3, CLSTN1, COL11A1, P4HA1, PDLIM4, ST5, FSTL1, PPP1R13L, PLTP, PDLIM7, and CALU.

Representative genes of subtype 3 (Sub3): MYH9, FLNA, P4HA2, LOXL2, FN1, CD55, FLT1, ECM1, CCDC80, TSKU, HTRA1, COL12A1, SPON2, and ANGPTL2.

Representative genes of subtype 4 (Sub4): PLEC, LPGAT1, NRDC, PRPF40A, CSDE1, IPO7, CDK1, HMGA1, DDX5, RASA1, ADSS, GMPS, CSE1L, PSME3, CAPRIN1, and BZW1.

Representative genes of subtype 5 (Sub5): HSPB6, HSPA12A, ANXA6, VIM, UCHL1, PRPH, MAP1B, CD81, ANK2, AKAP12, ITSN1, RTN1, COL28A1, KCTD12, SPON1, SYNPO2, and EPB41L3.

Representative genes of subtype 6 (Sub6): CTNND2, DTNA, REG1A, PRSS2, CPA1, CPB1, ACAT1, CPA2, PNLIPRP1, PRDX4, SNTB1, PDCD4, CTRC, FKBP11, and SEC11C.

Representative genes of all subtypes (All Sub) of pancreatic ductal adenocarcinoma: KRT19, RAB27B, QSOX1, VILL, GNPAT, ABCC3, GP2, ETHE1, BPNT1, AGR2, PIGR, SRC, CTSE, JUP, RPL7, TSPAN8, SRM, VDAC1, SCP2, RPS3, AK4, RPL9, RDX, RPL3, RPL13A, RPL5, RPS9, HK2, RAB25, GNG2, RPL15, RPL37, RPS7, RPL8, RPL18A, RPL6, PABPC4, INF2, SLC25A24, MYH14, GALNT7, GOLM1, MCU, GSDMB, CYP2S1, HTATIP2, SDCBP2, SYTL2, PREB, MYO6, PKP3, SNTB2, and S100A11.

The 150 identified subtype-representative gene peptide samples were mixed together to form a subtype-representative peptide sample, which was to be mixed with each pancreatic cancer patient-derived peptide sample to determine the subtype of the pancreatic cancer patient. In this case, the pressure cycling technology-based Barocycler system was used to obtain each pancreatic cancer patient-derived peptide sample. First, an ultra-high pressure of 45,000 psi and a low pressure of 15 psi were alternately applied to a microtube containing a tissue sample and a dissolution buffer to effectively disrupt the cell wall, followed by protein extraction. Then, Lys-C and trypsin, which are digestive enzymes, were added to perform protein digestion, and an ultra-high pressure of 20,000 psi and a low pressure of 15 psi were alternately applied, thereby obtaining peptide samples from a total of 16 pancreatic cancer tissue samples within 3 hours. Next, the obtained peptide sample from the pancreatic cancer patient was subjected to a C18 spin column-based desalting process, and then subjected to BCA quantification, thereby obtaining a pancreatic cancer patient-derived peptide sample containing quantitative information.

Next, in order to determine the subtype of the pancreatic cancer patient, the patient-derived sample was mixed with the subtype-representative peptide sample containing information on the 150 subtype-representative genes to construct a peptide sample for subtype determination. As top-3 transition capable of reproducible and stable MRM analysis for each peptide, y-ions with a charge state of +2 or +3 were selected.

Subtype information, gene symbols, and protein names for all 150 subtype-representative peptides are shown in Table 1 below.

TABLE 1

| Subtype | Gene | |
|---|---|---|
| Information | Symbol | Protein name |
| Sub1 | CLDN18 | claudin 18 |
| Sub1 | EPS8L3 | phosphatase and actin regulator 2 |
| Sub1 | CAPN5 | calpain 5 |
| Sub1 | GMDS | GDP-mannose 4,6-dehydratase |
| Sub1 | BCAS1 | breast carcinoma amplified sequence 1 |
| Sub1 | IDH1 | Isocitrate dehydrogenase [NADP] |
| Sub1 | DDAH1 | dimethylarginine |
| | | dimethylaminohydrolase 1 |
| Sub1 | SOD1 | superoxide dismutase 1 |
| Sub1 | VIL1 | villin 1 |
| Sub1 | GPX2 | glutathione peroxidase 2 |
| Sub1 | AOC1 | amine oxidase, copper containing 1 |
| Sub1 | LGALS4 | galectin 4 |
| Sub1 | MICU2 | mitochondrial calcium uptake 2 |
| Sub1 | POF1B | premature ovarian failure, 1B |
| Sub1 | MICU1 | mitochondrial calcium uptake 1 |
| Sub1 | PLS1 | plastin 1 |
| Sub1 | BDH1 | 3-hydroxybutyrate dehydrogenase 1 |
| Sub2 | UNC5B | unc-5 netrin receptor B |
| Sub2 | PPP1R3G | protein phosphatase 1 regulatory |
| | | subunit 3G |
| Sub2 | IGFBP3 | insulin like growth factor binding protein 3 |
| Sub2 | EDIL3 | EGF like repeats and discoidin domains 3 |
| Sub2 | CLSTN1 | Calsyntenin-1 |
| Sub2 | COL11A1 | collagen type XI alpha 1 chain |
| Sub2 | P4HA1 | prolyl 4-hydroxylase subunit alpha 1 |
| Sub2 | PDLIM4 | PDZ and LIM domain 4 |
| Sub2 | ST5 | DENN domain-containing protein 2B |
| Sub2 | FSTL1 | Follistatin-related protein 1 |
| Sub2 | PPP1R13L | RelA-associated inhibitor |
| Sub2 | PLTP | phospholipid transfer protein |
| Sub2 | PDLIM7 | PDZ and LIM domain protein 7 |
| Sub2 | CALU | Calumenin |
| Sub3 | MYH9 | myosin heavy chain 9 |
| Sub3 | FLNA | Filamin-A |
| Sub3 | P4HA2 | prolyl 4-hydroxylase subunit alpha 2 |
| Sub3 | LOXL2 | lysyl oxidase like 2 |
| Sub3 | FN1 | fibronectin 1 |
| Sub3 | CD55 | CD55 molecule (Cromer blood group) |
| Sub3 | FLT1 | fms related tyrosine kinase 1 |
| Sub3 | ECM1 | extracellular matrix protein 1 |
| Sub3 | CCDC80 | coiled-coil domain containing 80 |
| Sub3 | TSKU | tsukushi, small leucine rich proteoglycan |
| Sub3 | HTRA1 | HtrA serine peptidase 1 |
| Sub3 | COL12A1 | collagen type XII alpha 1 chain |
| Sub3 | SPON2 | spondin 2 |
| Sub3 | ANGPTL2 | angiopoietin like 2 |
| Sub4 | PLEC | Plectin |
| Sub4 | LPGAT1 | Acyl-CoA: lysophosphatidylglycerol |
| | | acyltransferase 1 |
| Sub4 | NRDC | Nardilysin |
| Sub4 | PRPF40A | Pre-mRNA-processing factor 40 |
| | | homologA |
| Sub4 | CSDE1 | Cold shock domain-containing protein E1 |
| Sub4 | IPO7 | Importin-7 |
| Sub4 | CDK1 | cyclin dependent kinase 1 |
| Sub4 | HMGA1 | high mobility group AT-hook 1 |
| Sub4 | DDX5 | Probable ATP-dependent RNA |
| | | helicaseDDX5 |
| Sub4 | RASA1 | Ras GTPase-activating protein 1 |
| Sub4 | ADSS | Adenylosuccinate synthetase isozyme 2 |
| Sub4 | GMPS | GMP synthase [glutamine-hydrolyzing] |
| Sub4 | CSE1L | Exportin-2 |
| Sub4 | PSME3 | proteasome activator subunit 3 |
| Sub4 | CAPRIN1 | Caprin-1 |
| Sub4 | BZW1 | Basic leucine zipper and W2 domain- |
| | | containing protein 1 |
| Sub5 | HSPB6 | heat shock protein family B (small) |
| | | member 6 |
| Sub5 | HSPA12A | heat shock protein family A (Hsp70) |
| | | member 12A |
| Sub5 | ANXA6 | Annexin A6 |
| Sub5 | VIM | Vimentin |
| Sub5 | UCHL1 | ubiquitin C-terminal hydrolase L1 |
| Sub5 | PRPH | peripherin |
| Sub5 | MAP1B | Microtubule-associated protein 1B |
| Sub5 | CD81 | CD81 molecule |
| Sub5 | ANK2 | ankyrin 2 |
| Sub5 | AKAP12 | A-kinase anchoring protein 12 |
| Sub5 | ITSN1 | Intersectin-1 |
| Sub5 | RTN1 | Reticulon-1 |
| Sub5 | COL28A1 | collagen type XXVIII alpha 1 chain |
| Sub5 | KCTD12 | BTB/POZ domain-containing |
| | | protein KCTD12 |
| Sub5 | SPON1 | spondin 1 |
| Sub5 | SYNPO2 | synaptopodin 2 |
| Sub5 | EPB41L3 | erythrocyte membrane protein |
| | | band 4.1 like 3 |
| Sub5 | AKAP12 | A-kinase anchoring protein 12 |
| Sub6 | CTNND2 | catenin delta 2 |
| Sub6 | DTNA | dystrobrevin alpha |
| Sub6 | REG1A | regenerating family member 1 alpha |
| Sub6 | PRSS2 | protease, serine 2 |
| Sub6 | CPA1 | carboxypeptidase A1 |
| Sub6 | CPB1 | carboxypeptidase B1 |
| Sub6 | ACAT1 | acetyl-CoA acetyltransferase 1 |
| Sub6 | CPA2 | carboxypeptidase A2 |
| Sub6 | PNLIPRP1 | pancreatic lipase related protein 1 |
| Sub6 | PRDX4 | peroxiredoxin 4 |
| Sub6 | SNTB1 | syntrophin beta 1 |
| Sub6 | PDCD4 | programmed cell death 4 |
| Sub6 | CTRC | chymotrypsin C |
| Sub6 | FKBP11 | FK506 binding protein 11 |
| Sub6 | SEC11C | SEC11 homolog C, signal peptidase |
| | | complex subunit |
| Sub6 | CPA1 | carboxypeptidase A1 |
| All Sub | KRT19 | Keratin, type I cytoskeletal 19 |
| All Sub | RAB27B | RAB27B, member RAS oncogene family |
| All Sub | QSOX1 | quiescin sulfhydryl oxidase 1 |
| All Sub | VILL | villin like |
| All Sub | GNPAT | Dihydroxyacetone phosphate |
| | | acyltransferase |
| All Sub | ABCC3 | ATP binding cassette subfamily C |
| | | member 3 |
| All Sub | GP2 | glycoprotein 2 |
| All Sub | ETHE1 | Persulfide dioxygenase ETHE1, |
| | | mitochondrial |
| All Sub | BPNT1 | 3′(2′), 5′-bisphosphate nucleotidase 1 |
| All Sub | AGR2 | anterior gradient 2, protein disulphide |
| | | isomerase family member |
| All Sub | PIGR | polymeric immunoglobulin receptor |
| All Sub | SRC | SRC proto-oncogene, non-receptor |
| | | tyrosine kinase |
| All Sub | CTSE | cathepsin E |
| All Sub | JUP | Junction plakoglobin |
| All Sub | RPL7 | ribosomal protein L7 |
| All Sub | TSPAN8 | tetraspanin 8 |
| All Sub | SRM | spermidine synthase |
| All Sub | VDAC1 | Voltage-dependent anion-selective channel |
| | | protein 1 |
| All Sub | SCP2 | sterol carrier protein 2 |
| All Sub | RPS3 | ribosomal protein S3 |
| All Sub | AK4 | adenylate kinase 4 |
| All Sub | RPL9 | 60S ribosomal protein L9 |
| All Sub | RDX | Radixin |
| All Sub | RPL3 | ribosomal protein L3 |
| All Sub | RPL13A | ribosomal protein L13a |
| All Sub | RPL5 | ribosomal protein L5 |
| All Sub | RPS9 | ribosomal protein S9 |
| All Sub | HK2 | hexokinase 2 |
| All Sub | RAB25 | RAB25, member RAS oncogene family |
| All Sub | GNG2 | Guanine nucleotide-binding protein |
| | | G(I)/G(S)/G(O) subunit gamma-2 |
| All Sub | RPL15 | ribosomal protein L15 |
| All Sub | RPL37 | ribosomal protein L37 |
| All Sub | RPS7 | 40S ribosomal protein S7 |
| All Sub | RPL8 | ribosomal protein L8 |
| All Sub | RPL18A | 60S ribosomal protein L18a |
| All Sub | RPL6 | ribosomal protein L6 |
| All Sub | PABPC4 | poly(A) binding protein cytoplasmic 4 |
| All Sub | INF2 | Inverted formin-2 |
| All Sub | SLC25A24 | Calcium-binding mitochondrial |
| | | carrier protein ScaMC-1 |
| All Sub | MYH14 | Myosin-14 |
| All Sub | GALNT7 | polypeptide |
| | | N-acetylgalactosaminyltransferase 7 |
| All Sub | GOLM1 | Golgi membrane protein 1 |
| All Sub | MCU | Calcium uniporter protein, mitochondrial |
| All Sub | GSDMB | gasdermin B |
| All Sub | CYP2S1 | Cytochrome P450 2S1 |
| All Sub | HTATIP2 | Oxidoreductase HTATIP2 |
| All Sub | SDCBP2 | syndecan binding protein 2 |
| All Sub | SYTL2 | synaptotagmin like 2 |
| All Sub | PREB | Prolactin regulatory element-binding |
| | | protein |
| All Sub | MYO6 | Unconventional myosin-VI |
| All Sub | PKP3 | plakophilin 3 |
| All Sub | SNTB2 | syntrophin beta 2 |
| All Sub | S100A11 | S100 calcium binding protein A11 |
| Mutation | KRAS | GTPase KRas |
| Mutation | KRAS | GTPase KRas |

1. A method for determining a subtype of pancreatic ductal adenocarcinoma, the method comprising the following steps (1) to (4):
(1) pulverizing a pancreatic ductal adenocarcinoma lesion tissue isolated from a pancreatic ductal adenocarcinoma patient;
(2) obtaining a peptide sample for the patient by extracting and digesting proteins from the lesion tissue;
(3) measuring expression levels of representative genes of pancreatic ductal adenocarcinoma subtypes 1 to 6 from the peptide sample for the patient, wherein the representative genes of pancreatic ductal adenocarcinoma subtypes 1 to 6 are each at least one selected from the group consisting of the following genes:
representative genes of subtype 1 (Sub1): CLDN18, EPS8L3, CAPN5, GMDS, BCAS1, IDH1, DDAH1, SOD1, VIL1, GPX2, AOC1, LGALS4, MICU2, POF1B, MICU1, PLS1, and BDH1;
representative genes of subtype 2 (Sub2): UNC5B, PPP1R3G, IGFBP3, EDIL3, CLSTN1, COL11A1, P4HA1, PDLIM4, ST5, FSTL1, PPP1R13L, PLTP, PDLIM7, and CALU;
representative genes of subtype 3 (Sub3): MYH9, FLNA, P4HA2, LOXL2, FN1, CD55, FLT1, ECM1, CCDC80, TSKU, HTRA1, COL12A1, SPON2, and ANGPTL2;
representative genes of subtype 4 (Sub4): PLEC, LPGAT1, NRDC, PRPF40A, CSDE1, IPO7, CDK1, HMGA1, DDX5, RASA1, ADSS, GMPS, CSE1L, PSME3, CAPRIN1, and BZW1;
representative genes of subtype 5 (Sub5): HSPB6, HSPA12A, ANXA6, VIM, UCHL1, PRPH, MAP1B, CD81, ANK2, AKAP12, ITSN1, RTN1, COL28A1, KCTD12, SPON1, SYNPO2, and EPB41L3; and
representative genes of subtype 6 (Sub6): CTNND2, DTNA, REG1A, PRSS2, CPA1, CPB1, ACAT1, CPA2, PNLIPRP1, PRDX4, SNTB1, PDCD4, CTRC, FKBP11, and SEC11C; and
(4) determining a subtype of the pancreatic ductal adenocarcinoma patient by comparing the expression levels of the representative genes of pancreatic ductal adenocarcinoma subtypes 1 to 6.

2. The method of claim 1, wherein the comparing the expression levels of the representative genes of pancreatic ductal adenocarcinoma subtypes 1 to 6 is performed by combining and comparing expression levels of genes most contributing to distinguishing subtypes 1 to 6 and the following all subtypes of pancreatic ductal adenocarcinoma from one another:
representative genes of all subtypes (All Sub) of pancreatic ductal adenocarcinoma: KRT19, RAB27B, QSOX1, VILL, GNPAT, ABCC3, GP2, ETHE1, BPNT1, AGR2, PIGR, SRC, CTSE, JUP, RPL7, TSPAN8, SRM, VDAC1, SCP2, RPS3, AK4, RPL9, RDX, RPL3, RPL13A, RPL5, RPS9, HK2, RAB25, GNG2, RPL15, RPL37, RPS7, RPL8, RPL18A, RPL6, PABPC4, INF2, SLC25A24, MYH14, GALNT7, GOLM1, MCU, GSDMB, CYP2S1, HTATIP2, SDCBP2, SYTL2, PREB, MYO6, PKP3, SNTB2, and S100A11.

3. The method of claim 1, wherein the pulverizing the pancreatic ductal adenocarcinoma lesion tissue in step (1) is performed by cryogenic pulverization.

4. The method of claim 1, wherein the extracting the proteins in step (2) is performed by pressure cycling technology.

5. The method of claim 1, wherein the representative genes of pancreatic ductal adenocarcinoma subtypes 1 to 6 in step (3) are those identified by performing proteogenomic analysis for a combination of mRNA data for pancreatic ductal adenocarcinoma lesion tissue samples with global proteome data and phosphoproteome data.

6. The method of claim 1 or 2, wherein the measuring and comparing the expression levels of the representative genes of all subtypes and subtypes 1 to 6 of pancreatic ductal adenocarcinoma comprises steps of:
constructing a stable isotope-labeled peptide panel representing the representative genes of all subtype of pancreatic ductal adenocarcinoma and the genes of each of subtypes 1 to 6;
mixing the peptide sample for the patient and the stable isotope-labeled peptide panel; and
determining the subtype of the pancreatic ductal adenocarcinoma patient by analyzing the mixture by quantitative mass spectrometry.

7. The method of claim 6, wherein the quantitative mass spectrometry is performed by comparing signal intensities of the patient-derived peptides with those of the stable isotope-labeled peptides.

8. The method of claim 7, wherein the ratio of the signal intensities is expressed as a signal-intensity contour map for each peptide with the peptide and the peptide elution time as two axes.

9. A kit for determining a subtype of pancreatic ductal adenocarcinoma, the kit comprising agents for measuring expression levels of representative genes of pancreatic ductal adenocarcinoma subtypes 1 to 6, wherein the representative genes of pancreatic ductal adenocarcinoma subtypes 1 to 6 are each at least one selected from the group consisting of the following genes:
representative genes of subtype 1 (Sub1): CLDN18, EPS8L3, CAPN5, GMDS, BCAS1, IDH1, DDAH1, SOD1, VIL1, GPX2, AOC1, LGALS4, MICU2, POF1B, MICU1, PLS1, and BDH1;
representative genes of subtype 2 (Sub2): UNC5B, PPP1R3G, IGFBP3, EDIL3, CLSTN1, COL11A1, P4HA1, PDLIM4, ST5, FSTL1, PPP1R13L, PLTP, PDLIM7, and CALU;
representative genes of subtype 3 (Sub3): MYH9, FLNA, P4HA2, LOXL2, FN1, CD55, FLT1, ECM1, CCDC80, TSKU, HTRA1, COL12A1, SPON2, and ANGPTL2;
representative genes of subtype 4 (Sub4): PLEC, LPGAT1, NRDC, PRPF40A, CSDE1, IPO7, CDK1, HMGA1, DDX5, RASA1, ADSS, GMPS, CSE1L, PSME3, CAPRIN1, and BZW1;
representative genes of subtype 5 (Sub5): HSPB6, HSPA12A, ANXA6, VIM, UCHL1, PRPH, MAP1B, CD81, ANK2, AKAP12, ITSN1, RTN1, COL28A1, KCTD12, SPON1, SYNPO2, and EPB41L3; and
representative genes of subtype 6 (Sub6): CTNND2, DTNA, REG1A, PRSS2, CPA1, CPB1, ACAT1, CPA2, PNLIPRP1, PRDX4, SNTB1, PDCD4, CTRC, FKBP11, and SEC11C.

10. The kit of claim 9, comprising an agent for measuring an expression level of at least one gene selected from the group consisting of the following representative genes of all subtypes of pancreatic ductal adenocarcinoma, which is compared with the expression levels of representative genes of subtypes 1 to 6:
representative genes of all subtypes (All Sub) of pancreatic ductal adenocarcinoma: KRT19, RAB27B, QSOX1, VILL, GNPAT, ABCC3, GP2, ETHE1, BPNT1, AGR2, PIGR, SRC, CTSE, JUP, RPL7, TSPAN8, SRM, VDAC1, SCP2, RPS3, AK4, RPL9, RDX, RPL3, RPL13A, RPL5, RPS9, HK2, RAB25, GNG2, RPL15, RPL37, RPS7, RPL8, RPL18A, RPL6, PABPC4, INF2, SLC25A24, MYH14, GALNT7, GOLM1, MCU, GSDMB, CYP2S1, HTATIP2, SDCBP2, SYTL2, PREB, MYO6, PKP3, SNTB2, and S100A11.

11. The kit of claim 9, wherein the agents for measuring the expression levels of the representative genes of all subtypes of pancreatic ductal adenocarcinoma and the representative genes of subtypes 1 to 6 comprise a stable isotope-labeled peptide panel representing the representative genes of all subtypes of pancreatic ductal adenocarcinoma and the representative genes of each of subtypes 1 to 6.

12. A method for predicting prognosis of a pancreatic ductal adenocarcinoma patient, the method comprising the following steps (1) to (5):
(1) pulverizing a pancreatic ductal adenocarcinoma lesion tissue isolated from a pancreatic ductal adenocarcinoma patient;
(2) obtaining a peptide sample for the patient by extracting and digesting proteins from the lesion tissue;
(3) measuring expression levels of representative genes of pancreatic ductal adenocarcinoma subtypes 1 to 6 from the peptide sample for the patient, wherein the representative genes of pancreatic ductal adenocarcinoma subtypes 1 to 6 are each at least one selected from the group consisting of the following genes:
representative genes of subtype 1 (Sub1): CLDN18, EPS8L3, CAPN5, GMDS, BCAS1, IDH1, DDAH1, SOD1, VIL1, GPX2, AOC1, LGALS4, MICU2, POF1B, MICU1, PLS1, and BDH1;
representative genes of subtype 2 (Sub2): UNC5B, PPP1R3G, IGFBP3, EDIL3, CLSTN1, COL11A1, P4HA1, PDLIM4, ST5, FSTL1, PPP1R13L, PLTP, PDLIM7, and CALU;
representative genes of subtype 3 (Sub3): MYH9, FLNA, P4HA2, LOXL2, FN1, CD55, FLT1, ECM1, CCDC80, TSKU, HTRA1, COL12A1, SPON2, and ANGPTL2;
representative genes of subtype 4 (Sub4): PLEC, LPGAT1, NRDC, PRPF40A, CSDE1, IPO7, CDK1, HMGA1, DDX5, RASA1, ADSS, GMPS, CSE1L, PSME3, CAPRIN1, and BZW1;
representative genes of subtype 5 (Sub5): HSPB6, HSPA12A, ANXA6, VIM, UCHL1, PRPH, MAP1B, CD81, ANK2, AKAP12, ITSN1, RTN1, COL28A1, KCTD12, SPON1, SYNPO2, and EPB41L3; and
representative genes of subtype 6 (Sub6): CTNND2, DTNA, REG1A, PRSS2, CPA1, CPB1, ACAT1, CPA2, PNLIPRP1, PRDX4, SNTB1, PDCD4, CTRC, FKBP11, and SEC11C;
(4) determining a subtype of the pancreatic ductal adenocarcinoma patient by comparing the expression levels of the representative genes of pancreatic ductal adenocarcinoma subtypes 1 to 6; and
(5) predicting prognosis based on the determined subtype.

13. The method of claim 12, wherein the expression levels of the representative genes of subtypes 1 to 6 are compared with an expression level of at least one gene selected from the group consisting of the following representative genes of all subtypes of pancreatic ductal adenocarcinoma:
representative genes of all subtypes (All Sub) of pancreatic ductal adenocarcinoma: KRT19, RAB27B, QSOX1, VILL, GNPAT, ABCC3, GP2, ETHE1, BPNT1, AGR2, PIGR, SRC, CTSE, JUP, RPL7, TSPAN8, SRM, VDAC1, SCP2, RPS3, AK4, RPL9, RDX, RPL3, RPL13A, RPL5, RPS9, HK2, RAB25, GNG2, RPL15, RPL37, RPS7, RPL8, RPL18A, RPL6, PABPC4, INF2, SLC25A24, MYH14, GALNT7, GOLM1, MCU, GSDMB, CYP2S1, HTATIP2, SDCBP2, SYTL2, PREB, MYO6, PKP3, SNTB2, and S100A11.

14. The method of claim 12, wherein subtypes 2 to 4 are predicted to have a poor prognosis compared to subtypes 1, 5 and 6.

15. The method of claim 2, wherein the measuring and comparing the expression levels of the representative genes of all subtypes and subtypes 1 to 6 of pancreatic ductal adenocarcinoma comprises steps of:
constructing a stable isotope-labeled peptide panel representing the representative genes of all subtype of pancreatic ductal adenocarcinoma and the genes of each of subtypes 1 to 6;
mixing the peptide sample for the patient and the stable isotope-labeled peptide panel; and
determining the subtype of the pancreatic ductal adenocarcinoma patient by analyzing the mixture by quantitative mass spectrometry.

16. The kit of claim 10, wherein the agents for measuring the expression levels of the representative genes of all subtypes of pancreatic ductal adenocarcinoma and the representative genes of subtypes 1 to 6 comprise a stable isotope-labeled peptide panel representing the representative genes of all subtypes of pancreatic ductal adenocarcinoma and the representative genes of each of subtypes 1 to 6.
